(19) United States
(12) Patent Application Publication
(10) Pub. No.: US 2024/0000356 A1
(43) Pub. Date: Jan. 4, 2024

(54) MEASUREMENT ADAPTER, MEASUREMENT SYSTEM, AND MEASUREMENT METHOD

(71) Applicant: TERUMO KABUSHKI KAISHA, Tokyo (JP)

(72) Inventor: Satoshi SAWADA, Hadano-shi (JP)

(73) Assignee: TERUMO KABUSHIKI KAISHA, Tokyo (JP)

(21) Appl. No.: 18/466,988

(22) Filed: Sep. 14, 2023

Related U.S. Application Data
(63) Continuation of application No. PCT/JP2022/010545, filed on Mar. 10, 2022.

(30) Foreign Application Priority Data
Mar. 31, 2021 (JP) 2021-061293

(51) Int. Cl. A61B 5/20; A61B 5/00; G01N 33/493; A61M 25/10

(52) U.S. Cl. CPC A61B 5/208; A61B 5/6853; G01N 33/493; A61M 25/10; A61M 2025/1068

(57) ABSTRACT

A measurement adapter includes a catheter connection portion connectable to a bladder indwelling catheter, a urine collection bag connection portion connectable to a urine collection bag, a flow path disposed between the catheter connection portion and the urine collection bag connection portion, and a plurality of sensor holding portions that holds sensors configured to detect a state of urine flowing in the flow path.

### CROSS-REFERENCES TO RELATED APPLICATIONS

This application is a continuation of International Application No. PCT/JP2022/010545 filed on Mar. 10, 2022, which claims priority to Japanese Application No. 2021-061293 filed on Mar. 31, 2021, the entire content of both of which is incorporated herein by reference.

### TECHNOLOGICAL FIELD

The present disclosure generally relates to a measurement adapter, a measurement system, and a measurement method.

### BACKGROUND DISCUSSION

Bladder indwelling catheters with various specifications are used in medical settings. A doctor selects and uses a bladder indwelling catheter with an appropriate specification on the basis of gender, age, a body type, a condition, and the like, of a patient.

U.S. Patent Application Publication No. 2020-0205718 A, discloses a bladder indwelling catheter equipped with an oxygen sensor has been proposed that by measuring an oxygen partial pressure in urine in real time, signs of acute kidney injury can be found relatively early.

However, in a case of using the bladder indwelling catheter of U.S. Patent Application Publication No. 2020-0205718 A, a doctor cannot select an appropriate bladder indwelling catheter from variations as in existing bladder indwelling catheters.

### SUMMARY

A measurement adapter is disclosed, which is capable of measuring a state of urine in combination with an existing bladder indwelling catheter.

A measurement adapter includes a catheter connection portion connectable to a bladder indwelling catheter, a urine collection bag connection portion connectable to a urine collection bag, a flow path disposed between the catheter connection portion and the urine collection bag connecting portion, and a plurality of sensor holding portions configured to hold sensors capable of detecting a state of urine flowing in the flow path.

A measurement system including a measurement adapter, the measurement adapter including: a catheter connection portion configured to be connectable to a bladder indwelling catheter; a urine collection bag connection portion configured to be connectable to a urine collection bag; a flow path disposed between the catheter connection portion and the urine collection bag connection portion; and a plurality of sensor holding portions configured to hold sensors configured to detect a state of urine flowing in the flow path; and a measurement device, the measurement device including: a data acquisition unit configured to acquire data from the sensors held by the sensor holding portions; and a display unit configured to display information on a patient in which the bladder indwelling catheter is indwelled on the basis of the acquired data.

A measurement method includes acquiring data acquired using sensors from a measurement adapter connected between a bladder indwelling catheter and a urine collection bag, the measurement adapter including a flow path through which urine flows from the bladder indwelling catheter to the urine collection bag, and a plurality of sensor holding portions that hold the sensors that detect a state of the urine flowing in the flow path; and displaying information on a state of a urinary organ of a patient in which the bladder indwelling catheter is indwelled on the basis of the acquired data

In one aspect, it is possible to provide a measurement adapter, or the like, capable of measuring a state of urine in combination with an existing bladder indwelling catheter.

### BRIEF DESCRIPTION OF THE DRAWINGS

FIG. 1 is an explanatory view for explaining a configuration of a measurement system.

FIG. 2 is a cross-sectional view of a measurement adapter.

FIG. 3 is a view taken along an arrow III in FIG. 2.

FIG. 4 is an explanatory view for explaining a structure of the measurement adapter.

FIG. 5 is an explanatory view for explaining a configuration of a measurement device.

FIG. 6 is a perspective view of a fastener.

FIG. 7 is a flowchart for explaining flow of processing of a program.

FIG. 8 is a screen example of Modification 1-1.

FIG. 9 is an explanatory view for explaining a configuration of Modification 1-2.

FIG. 10 is an explanatory view for explaining a configuration of Modification 1-3.

FIG. 11 is an explanatory view for explaining a structure of a measurement adapter of Modification 1-4.

FIG. 12 is an explanatory view for explaining a method of using a measurement system of Modification 1-4.

FIG. 13 is an explanatory view for explaining a structure of a measurement adapter of a second embodiment.

FIG. 14 is an explanatory view for explaining a configuration of a measurement system of the second embodiment.

FIG. 15 is an enlarged view of a portion XV in FIG. 14.

FIG. 16 is a cross-sectional view for explaining a configuration of a distal portion of an optical fiber of Modification 2-1.

FIG. 17 is a cross-sectional view for explaining a configuration of a distal portion of an optical fiber of Modification 2-2.

FIG. 18 is an explanatory view for explaining a configuration of a measurement device of a third embodiment.

FIGS. 19A to 19C are time charts for explaining operation of the measurement device of the third embodiment.

FIGS. 20A to 20C are time charts for explaining operation of a measurement device of a fourth embodiment.

FIG. 21 is an explanatory view for explaining a configuration of a measurement device of a fifth embodiment.

FIGS. 22A to 22C are time charts for explaining operation of the measurement device of the fifth embodiment.

FIG. 23 is an explanatory view for explaining a configuration of a measurement device of a sixth embodiment.

FIG. 24 is a front view of a measurement adapter of a seventh embodiment.

FIG. 25 is a partial cross-sectional view taken along a line XXV-XXV in FIG. 24.

FIG. 26 is an explanatory view for explaining a configuration of an optical sensor unit.

FIG. 27 is an explanatory view for explaining a configuration of a measurement adapter of the seventh embodiment.

FIG. 28 is an explanatory view for explaining a configuration of an optical sensor unit of Modification 7-1.

FIG. 29 is an explanatory view for explaining a configuration of an optical sensor unit of Modification 7-2.

FIG. 30 is an explanatory view for explaining a configuration of an optical sensor unit of Modification 7-3.

FIG. 31 is a cross-sectional view of a measurement adapter of Modification 7-4.

FIG. 32 is a perspective view of a measurement adapter of Modification 7-5.

FIG. 33 is a functional block diagram of a measurement system of an eighth embodiment.

### DETAILED DESCRIPTION

Set forth below with reference to the accompanying drawings is a detailed description of embodiments of a measurement adapter, a measurement system, and a measurement method. In the drawings, similar components are denoted by the same reference signs, and the detailed description of the similar components will be appropriately omitted.

### First Embodiment

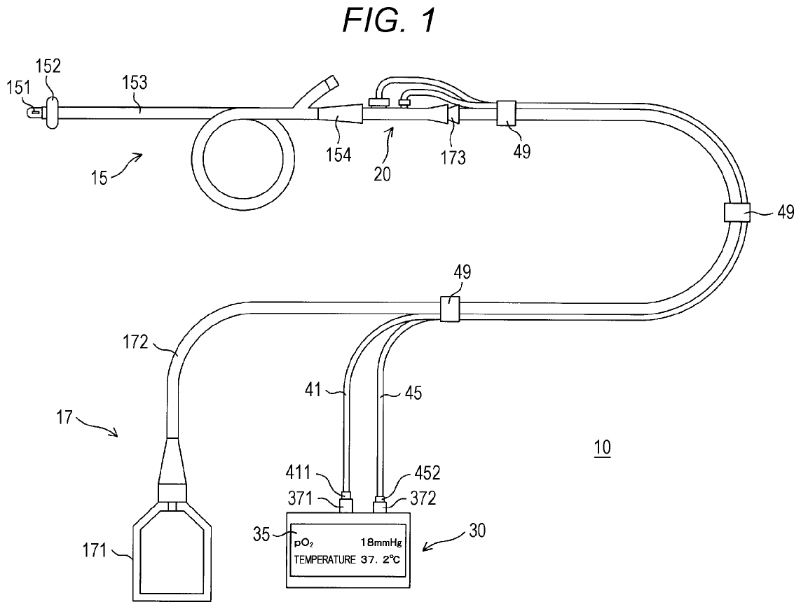

FIG. 1 is an explanatory view for explaining a configuration of a measurement system 10. The measurement system 10 can include a bladder indwelling catheter 15, a urine collection bag 17, a measurement adapter 20, and a measurement device 30. Note that FIG. 1 is a view schematically illustrating each component of the measurement system 10.

The bladder indwelling catheter 15 can include a shaft 153 having a side hole 151 and a balloon 152 at a distal end of the shaft 153, and a urination funnel 154 connected to one end (i.e., proximal end) of the shaft 153. The urine collection bag 17 can include a bag 171, a urine collection tube 172, and a connection tube 173. The bladder indwelling catheter 15 and the urine collection bag 17 of the present embodiment have been used in related art in a medical setting. Outline of a method of using the bladder indwelling catheter 15 and the urine collection bag 17 in related art will be described.

A user such as a doctor inserts the shaft 153 into a urethra of a patient after connecting the urination funnel 154 and the connection tube 173. In a state where the distal end of the shaft 153 enters the inside of the bladder, the user inflates the balloon 152. The balloon 152 illustrated in FIG. 1 is in an inflated state. By inflating the balloon 152, the shaft 153 does not come out of the urethra. The urine of the patient passes through the side hole 151, the shaft 153, and the urine collection tube 172 and is accumulated in the bag 171.

In the present embodiment, as illustrated in FIG. 1, the measurement adapter 20 is connected between the urination funnel 154 and the connection tube 173. The urine of the patient passes through the side hole 151, the shaft 153, the measurement adapter 20, and the urine collection tube 172 and is accumulated in the bag 171.

The measurement device 30 can include a display unit 35, a first connector 371, and a second connector 372. In the example illustrated in FIG. 1, an oxygen partial pressure (pO2) in the urine of the patient and a temperature of the urine flowing in the measurement adapter 20 are displayed on the display unit 35 in real time.

An optical fiber 41 and a thermocouple 45 are connected to the measurement adapter 20. The thermocouple 45 is an example of a temperature sensor. The optical fiber 41 and the thermocouple 45 can be fixed to the urine collection tube 172 at three positions by the fasteners 49. An optical fiber connector 411 provided at an end portion of the optical fiber 41 is connected to the first connector 371. A thermocouple connector 452 provided at an end portion of the thermocouple 45 is connected to the second connector 372.

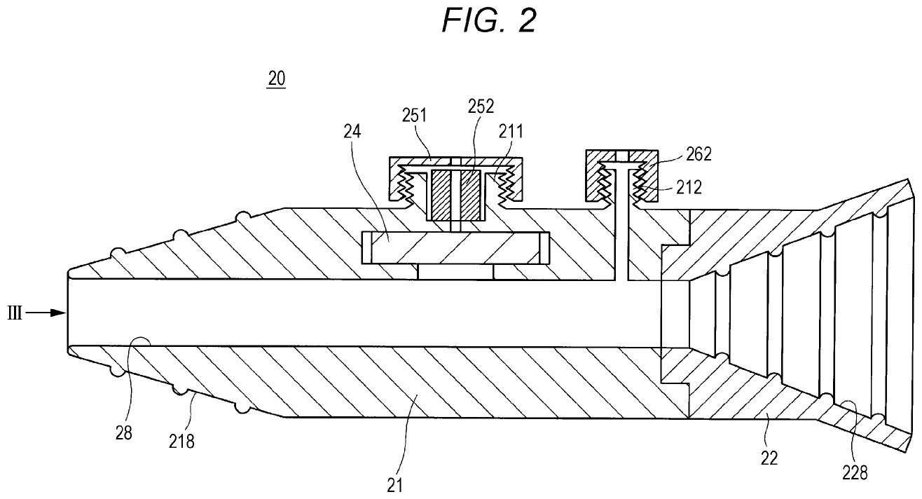

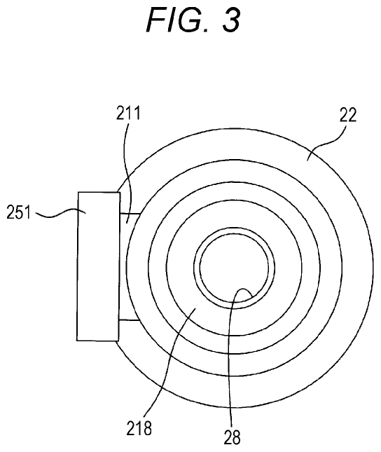

FIG. 2 is a cross-sectional view of the measurement adapter 20. FIG. 3 is a view taken along an arrow III in FIG. 2. The measurement adapter 20 can include a first tubular portion 21 and a second tubular portion 22. The first tubular portion 21 and the second tubular portion 22 are connected in a longitudinal direction. A flow path 28, which is a through-hole, is provided along the longitudinal direction of the measurement adapter 20. Urine that has entered the inside of the shaft 153 from the side hole 151 passes through the flow path 28 and enters the urine collection tube 172.

The first tubular portion 21 can have a substantially cylindrical shape and includes a first connection portion 211 and a second connection portion 212 protruding to a side surface. A male screw is provided on an outer periphery of each of the first connection portion 211 and the second connection portion 212. The first connection portion 211 and the second connection portion 212 are provided with through-holes penetrating to the flow path 28. The first connection portion 211 and the second connection portion 212 are arranged side by side along the longitudinal direction of the flow path 28.

A expanding outward is provided at an end portion on an outer peripheral side of the through-hole provided in the first connection portion 211. A cylindrical holding rubber 252 is inserted inside the step. A large-diameter portion is provided on a side close to the flow path 28 in the through-hole. A light emitter 24 is stored in the large-diameter portion. The light emitter 24 comes into contact with urine flowing through the flow path 28. An edge of the light emitter 24 is water-tightly bonded and fixed to the first tubular portion 21, and urine can be prevented from leaking from a hole provided in the first connection portion 211. The light emitter 24 will be described in detail later.

The first tubular portion 21 has a catheter connection portion 218 having a tapered shape on an outer surface of one end. The catheter connection portion 218 is provided with protrusions in a stripe or circular shape for retaining (i.e., engaging) the urination funnel 154. The first tubular portion 21 is relatively hard and can be made of, for example, a hard plastic. The catheter connection portion 218 has a size and a shape that can be connected to the urination funnel 154 of the bladder indwelling catheter 15, similarly to the connection tube 173 of the urine collection bag 17 used in related art.

The second tubular portion 22 has a urine collection bag connection portion 228 having a tapered shape expanding forward on an inner surface of one end. On a surface of the urine collection bag connection portion 228, protrusions are provided in a stripe or circular shape for retaining (i.e., engaging) the connection tube 173 of the urine bag 17. The second tubular portion 22 is made of a rubber or an elastomer and has rubber elasticity. The material constituting the second tubular portion 22 can be, for example, styrene-based, olefin-based, or polyester-based elastomer. Similarly, to the urination funnel 154 of the bladder indwelling catheter 15 in related art, the urine collection bag connection portion 228 has a size and a shape that can be connected to the connection tube 173 of the urine collection bag 17.

The first tubular portion 21 and the second tubular portion 22 are more desirably made of the same material as the material used for the connection portion between the existing bladder indwelling catheter 15 and the urine collection bag 17. Silicone rubber elastomer, urethane rubber elastomer, and the like can be used for these existing instruments.

Thus, the measurement adapter 20 can be attached between the bladder indwelling catheter 15 and the urine collection bag 17 used in related art. The user can use the bladder indwelling catheter 15 and the urine collection bag 17 appropriately selected according to the patient's condition, or the like, in combination with the measurement adapter 20.

The first tubular portion 21 and the second tubular portion 22 can be water-tightly fixed by, for example, adhesion or screwing. The first tubular portion 21 and the second tubular portion 22 may be formed by integral molding of different materials.

A first cap 251 having a through-hole on a top surface is attached to the first connection portion 211. A female screw to be screwed with the first connection portion 211 is provided on an inner surface of the first cap 251. A second cap 262 having a through-hole on a top surface is attached to the second connection portion 212. A female screw to be screwed with the second connection portion 212 is provided on an inner surface of the second cap 262. In FIGS. 2 and 3, the screws of the first cap 251 and the second cap 262 are loosened.

The light emitter 24 can be, for example, a plate made of a translucent resin into which a phosphor is kneaded. The light emitter 24 may be a translucent plate coated with a phosphor. In the present embodiment, a case of using a phosphor that emits fluorescence in response to oxygen in urine will be described as an example. Fluorescence is an example of radiation light to be emitted by the light emitter 24. By analyzing characteristics of the fluorescence emitted from the phosphor, an oxygen partial pressure and an oxygen concentration in the urine can be measured in real time.

Outline of a measurement method using a phosphor will be described. In a case where the phosphor is irradiated with excitation light, the phosphor is in an excited state with relatively high energy. Fluorescence is emitted from the phosphor in the excited state, and the phosphor returns to a ground state. The characteristics of the fluorescence such as intensity, a phase angle, and decay time of the emitted fluorescence change on the basis of a concentration of a quencher with which the phosphor is in contact and an environment such as an ambient temperature and an atmospheric pressure. Thus, by analyzing the characteristics of the radiation light, the concentration of the quencher, the ambient temperature, and the like, can be measured.

The quencher can be, for example, oxygen. For example, the quencher in contact with the phosphor, that is, a component to be measured can be selected by a diffusion osmosis membrane disposed on a surface of the light emitter 24. A phosphor that reacts with a specific quencher may be used. The radiation light emitted from the phosphor also includes phosphorescence. The measurement may be performed by analyzing the characteristics of the phosphorescence.

The light emitter 24 may have a phosphor only in a portion facing the flow path 28. The light emitter 24 may have a protrusion in a portion facing the flow path 28. By having the protrusion, the light emitter 24 reliably comes into contact with the urine flowing in the flow path 28.

The phosphor may emit fluorescence in response to carbon dioxide in the urine. By analyzing the characteristics of fluorescence, a partial pressure of carbon dioxide and a concentration of carbon dioxide in the urine can be measured in real time. The characteristics of the fluorescence to be emitted by the phosphor may change in accordance with a hydrogen ion index of the urine. By analyzing the characteristics of the fluorescence, the hydrogen ion index of the urine, that is, a potential of hydrogen (pH) (i.e., acidity or alkalinity) can be measured in real time.

The phosphor may emit fluorescence in response to ions such as potassium ions or sodium ions in urine. By analyzing the characteristics of the fluorescence, a concentration of an electrolyte in the urine can be measured in real time. In addition, a phosphor that reacts with an arbitrary component in urine to emit fluorescence may be used.

As described above, the characteristics of the fluorescence of the phosphor changes depending on the temperature. By analyzing the characteristics of the fluorescence, the temperature of the urine can be measured in real time. A light-emitting state of the phosphor may change depending on a flow rate of the urine to be contacted. By analyzing the characteristics of the fluorescence, the flow rate of the urine can be measured in real time.

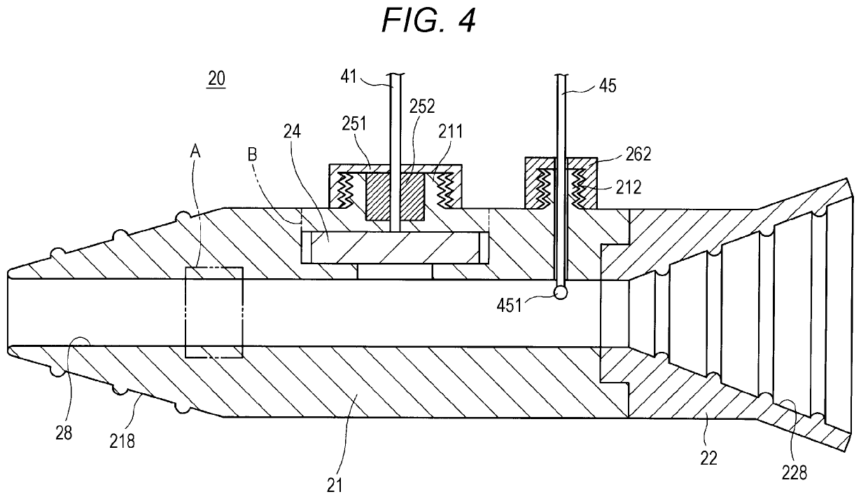

FIG. 4 is an explanatory view illustrating a structure of the measurement adapter 20. In FIG. 4, a distal end of the optical fiber 41 is inserted into a through-hole provided in the first connection portion 211, and a distal end of the thermocouple 45 is inserted into a through-hole provided in the second connection portion 212.

The distal end of the optical fiber 41 is abutted against the light emitter 24. The first cap 251 is tightened, and the holding rubber 252 is compressed in a vertical direction in FIG. 4. The compressed holding rubber 252 bulges in a radial direction, that is, a left-right direction in FIG. 4 and presses and fixes a side surface of the optical fiber 41.

By loosening the first cap 251, the holding rubber 252 returns to its original shape, and the optical fiber 41 becomes detachable from the measurement adapter 20. In other words, the optical fiber 41 is detachable from the measurement adapter 20.

A temperature measuring contact 451 provided at the distal end of the thermocouple 45 is disposed inside the flow path 28. The second cap 262 is tightened, and the thermocouple 45 is liquid-tightly held by a liquid-tight rubber.

The through-hole provided in the first connection portion 211 and the through-hole provided in the second connection portion 212 are both examples of sensor holding portions of the present embodiment. Among the through-holes provided in the first connection portion 211, a large-diameter portion in which the light emitter 24 is stored is an example of a light emitter holding portion of the present embodiment. Among the through-holes provided in the first connection portion 211, the holding rubber 252 arranged in the outer stepped portion and the stepped portion is an example of an optical fiber holding portion of the present embodiment. The through-hole provided in the second connection portion 212 is an example of a temperature sensor holding portion.

The first tubular portion 21 can include, for example, two members including a member in which a large-diameter portion in which the light emitter 24 is stored is opened to a surface and a stepped tubular member including the first connection portion 211. A boundary between the two members is indicated by a two-dot chain line with a symbol B in FIG. 4. The first tubular portion 21 can be manufactured by disposing the light emitter 24 at the bottom of the large-diameter portion and then bonding and fixing the stepped tubular member.

Structures for fixing the optical fiber 41 and the thermocouple 45 described using FIGS. 2 to 3 are all examples. Any structure that can help prevent misalignment of the optical fiber 41 and the thermocouple 45 during use of the measurement adapter 20 can be employed. For example, irregularities formed on surfaces of the optical fiber 41 and the thermocouple 45 may be fitted to irregularities formed on the measurement adapter 20.

In a case where it is not necessary to attach and detach the optical fiber 41 and the measurement adapter 20, the optical fiber and the measurement adapter may be fixed to each other using an adhesive. In a case where it is not necessary to attach and detach the thermocouple 45 and the measurement adapter 20, the thermocouple 45 and the measurement adapter 20 may be fixed in a state where liquid-tightness can be secured using an adhesive. It is possible to provide the measurement adapter 20 which can be rather easily set.

The through-hole provided in the first connection portion 211 and the through-hole provided in the second connection portion 212 may be inclined with respect to the flow path 28. In this way, the optical fiber 41 and the thermocouple 45 are attached obliquely to the measurement adapter 20. For example, in a case where the optical fiber 41 and the thermocouple 45 are attached to the measurement adapter 20 in a state of being inclined toward the urine collection bag 17 side, the optical fiber 41 and the thermocouple 45 are rather easily disposed along the urine collection tube 172. It is therefore possible to provide the measurement adapter 20 in which the optical fiber 41 and the thermocouple 45 are hardly detached.

A check valve for preventing backflow of the urine in the flow path 28 may be provided at a portion indicated by A in FIG. 4, that is, on a side closer to the catheter connection portion 218 than any of the sensor holding portions. By preventing backflow of the urine from the bag 171 into the bladder, a risk of occurrence of urinary tract infection can be reduced.

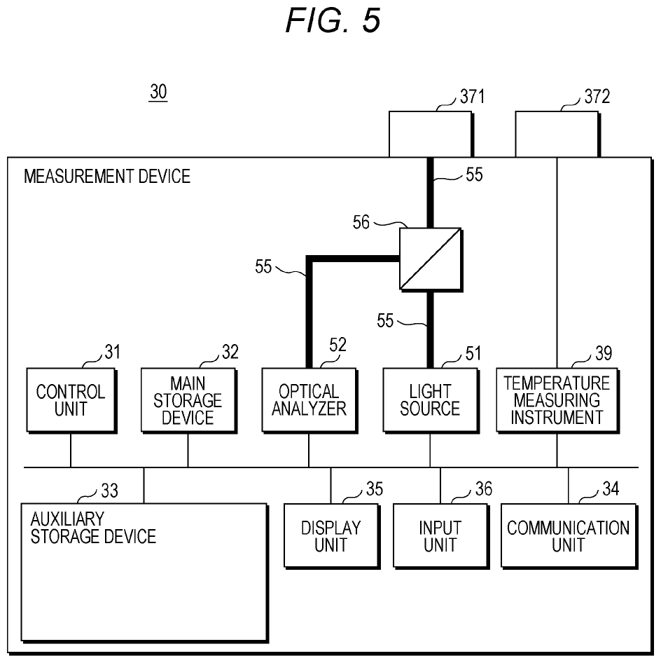

FIG. 5 is an explanatory view illustrating a configuration of the measurement device 30. In addition to the display unit 35, the first connector 371, and the second connector 372, the measurement device 30 can include a control unit 31, a main storage device 32, an auxiliary storage device 33, a communication unit 34, an input unit 36, a temperature measuring instrument 39, a light source 51, an optical analyzer 52, a light guide path 55, a beam splitter 56, and a bus. The control unit 31 can be an arithmetic control device that executes a program of the present embodiment. For the control unit 31, one or a plurality of central processing units (CPUs), graphics processing units (GPUs), multi-core CPUs, or the like, can be used. The control unit 31 is connected to each hardware unit constituting the measurement device 30 via a bus.

The main storage device 32 is a storage device such as a static random access memory (SRAM), a dynamic random access memory (DRAM), or a flash memory. In the main storage device 32, necessary information in the middle of processing to be performed by the control unit 31 and a program that is being executed by the control unit 31 are temporarily stored.

The auxiliary storage device 33 can be a storage device, for example, such as an SRAM, a flash memory, a hard disk, or a magnetic tape. The auxiliary storage device 33 stores a program to be executed by the control unit 31 and various kinds of data necessary for executing the program. The communication unit 34 is an interface that performs communication between the measurement device 30 and a network or another device.

The display unit 35 can be, for example, a liquid crystal display panel, an organic electro-luminescence (EL) panel, or the like. As illustrated in FIG. 1, the display unit 35 is attached to a chassis of the measurement device 30. The display unit 35 may be a display device separate from the measurement device 30. For example, a screen of another device such as a biological information monitor may also serve as the display unit 35.

The input unit 36 can be, for example, a button, or the like, provided on the chassis of the measurement device 30. The display unit 35 and the input unit 36 may be an integrated panel. The first connector 371 is an optical connector to which the optical fiber 41 is to be connected. The second connector 372 can be an electric connector to which the thermocouple 45 is to be connected. The measurement device 30 may include a plurality of first connectors 371.

The light source 51 can be, for example, a light emitting diode (LED) or a laser diode. The light source 51 irradiates the light emitter 24 with excitation light to excite the phosphor included in the light emitter 24. The excitation light is an example of the irradiation light with which the light emitter 24 is irradiated from the light source 51. The light emitted from the light source 51 include a part of the wavelength of the fluorescence emitted from the phosphor.

The optical analyzer 52 converts the received light into an electrical signal by, for example, a photodiode, and then performs analysis. The light guide path 55 connects between the light source 51 and the beam splitter 56, between the optical analyzer 52 and the beam splitter 56, and between the beam splitter 56 and the first connector 371.

The temperature measuring instrument 39 is connected to the second connector 372. The temperature measuring instrument 39 measures a temperature around the temperature measuring contact 451 on the basis of a thermoelectromotive force generated in the thermocouple 45 connected to the second connector 372 and outputs the measured temperature to the bus. Temperature measurement by the thermocouple 45 has been performed in related art, and thus, detailed description of the temperature measurement by the thermocouple 45 will be omitted. Instead of the thermocouple 45, any sensor capable of measuring a temperature, such as a thermistor, a resistance temperature detector, or a phosphor, may be connected to the temperature measuring instrument 39.

An optical filter that transmits only a wavelength region necessary for exciting the phosphor may be provided in the middle of the light guide path 55 or at an end portion of the light guide path 55 disposed between the light source 51 and the beam splitter 56. Even in a case where the light source 51 having a wide wavelength range is used, the wavelength range of the excitation light with which the light emitter 24 is irradiated can be precisely selected. Noise due to wavelengths other than the excitation light does not occur, so that it is possible to provide the measurement device 30 with relatively high measurement accuracy.

An optical lens may be disposed in the middle of the light guide path 55 or at an end portion of the light guide path 55. By effectively using the excitation light and the fluorescence, it is possible to provide the measurement device 30 with relatively high measurement sensitivity.

The measurement device 30 may include a second light source that supplies reference light to the optical analyzer 52 in addition to the light source 51 that emits light for excitation light. It is possible to provide the measurement device 30 that performs analysis using the reference light. The reference light emitted from the second light source is directly incident on the optical analyzer 52. The second light source and the optical analyzer 52 can be connected by, for example, a dedicated light guide path. A space between the second light source and the optical analyzer 52 may be a cavity configured such that light emitted from the second light source is incident on the optical analyzer 52.

The measurement device 30 may include a plurality of second connectors 372 and the temperature measuring instrument 39. The measurement device 30 may include a measuring instrument such as a flow meter or a pressure gauge, and a connector that connects a sensor corresponding to the measuring instrument.

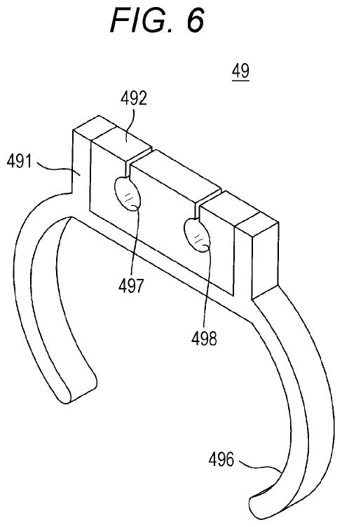

FIG. 6 is a perspective view of the fastener 49. The fastener 49 includes a first component 491 and a second component 492. The first component 491 can have a substantially C-shaped urine collection tube holding portion 496. The second component 492 can be attached to the outside of the urine collection tube holding portion 496. Each of the second components 492 can include an optical fiber holding portion 497 and a thermocouple holding portion 498 disposed at the bottom of a slit.

The urine collection tube holding portion 496 has a size that can be fitted to the outer periphery of the urine collection tube 172. The optical fiber holding portion 497 has a dimension capable of holding the optical fiber 41 pushed in from the slit. The thermocouple holding portion 498 has a size capable of holding the thermocouple 45 pushed in from the slit.

A material constituting the first component 491 is desirably a material that is relatively hard and rather easily bendable, such as a hard plastic. A material constituting the second component 492 is desirably an elastomer such as rubber. The material constituting the first component 491 may be, for example, a material softer than the material constituting the second component 492. The first component 491 and the second component 492 may be integrally formed of the same material.

As described with reference to FIG. 1, the optical fiber 41 and the thermocouple 45 can be, for example, fixed to the urine collection tube 172 at three positions by the fasteners 49. The optical fiber 41 and the thermocouple 45 may be fixed using, for example, a medical tape, or the like, instead of the fastener 49. The optical fiber 41 and the thermocouple 45 may be fixed, for example, to any place such as a drip stand or a bed fence.

Outline of a method of using the measurement system 10 will be described with reference to FIG. 1. The user connects the bladder indwelling catheter 15, the measurement adapter 20, and the urine collection bag 17. The user connects the optical fiber 41 and the thermocouple 45 to the measurement adapter 20 and the measurement device 30, respectively. The user fixes the optical fiber 41 and the thermocouple 45 to the urine collection tube 172 using the fastener 49.

The user inserts the shaft 153 into the urethra of the patient. In a state where the distal end of the shaft 153 enters the inside of the bladder, the user inflates the balloon 152. As described above, the bladder indwelling catheter 15 is indwelled in the patient. The urine of the patient passes through the side hole 151, the shaft 153, the flow path 28, and the urine collection tube 172 and is accumulated in the bag 171.

The description will be continued using FIG. 5. The user operates the measurement device 30 to operate the light source 51. The light emitter 24 is irradiated with the excitation light emitted from the light source 51 through the light guide path 55, the beam splitter 56, and the optical fiber 41. In a case where the light emitter 24 comes into contact with the urine flowing through the flow path 28, fluorescence corresponding to oxygen in the urine can be emitted. In other words, the light emitter 24 functions as a sensor capable of detecting an oxygen partial pressure and an oxygen concentration in the urine.

The fluorescence is incident on the beam splitter 56 via the optical fiber 41 and the light guide path 55. In other words, the optical fiber 41 has a function of propagating light emitted from the light source 51 to the light emitter 24 and light emitted from the light emitter 24. The fluorescence is incident on the light guide path 55 connected to the optical analyzer 52 by the beam splitter 56. The optical analyzer 52 analyzes characteristics of the incident fluorescence and outputs an oxygen partial pressure or an oxygen concentration in the urine to the bus in real time.

The control unit 31 displays the temperature output from the temperature measuring instrument 39 and the oxygen partial pressure in the urine output from the optical analyzer 52 on the display unit 35.

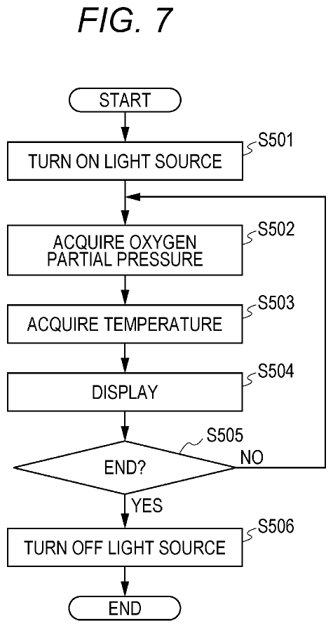

FIG. 7 is a flowchart illustrating flow of processing of the program. The control unit 31 starts the program of FIG. 7 in a case where the user gives an instruction to operate the light source 51.

The control unit 31 turns ON the light source 51 (S501). The light emitter 24 is irradiated with excitation light via the beam splitter 56 and the optical fiber 41. The fluorescence emitted from the phosphor of the light emitter 24 is incident on the optical analyzer 52 via the optical fiber 41 and the beam splitter 56. The optical analyzer 52 outputs urinary oxygen partial pressure data on the basis of the fluorescence. The control unit 31 acquires the urinary oxygen partial pressure data from the optical analyzer 52 (S502).

The temperature measuring instrument 39 outputs temperature data on the basis of a thermoelectromotive force of the thermocouple 45. The control unit 31 acquires the temperature data from the temperature measuring instrument 39 (S503). By S502 and S503, the control unit 31 implements a function of a data acquisition unit that acquires data from the sensors held in the sensor holding portions.

As exemplified in FIG. 1, the control unit 31 displays the urinary oxygen partial pressure and the temperature on the display unit 35 (S504). The control unit 31 determines whether or not to end the processing (S505). For example, the control unit 31 determines to end the processing in a case where operation to turn OFF the light source 51 is received, in a case where the optical fiber 41 is detached from the first connector 371, or in a case where the thermocouple 45 is detached from the second connector 372.

In a case where it is determined to end the processing (Yes in S505), the control unit 31 turns OFF the light source 51 (S506). The control unit 31 ends the processing. In a case where it is determined not to end the processing (No in S505), the control unit 31 returns to S502.

According to the present embodiment, it is possible to provide the measurement adapter 20 capable of measuring an oxygen partial pressure in urine, and the like, in real time in combination with the existing bladder indwelling catheter 15 and urine collection bag 17. The user can use the bladder indwelling catheter 15 selected on the basis of the patient's condition, past experience, expertise, and the like, in combination with the measurement adapter 20 of the present embodiment.

By measuring the oxygen partial pressure in the urine in real time, signs, for example, leading to acute kidney injury can be found rather early. As compared with the method in related art using a urine amount and a serum creatine level, or biomarkers, acute kidney injury can be found at a relatively early stage and appropriate treatment can be performed.

The control unit 31 may give a notification to the user, for example, in a case where the oxygen partial pressure in the urine becomes equal to or lower than a threshold value. For example, the control unit 31 notifies the user by, for example, display on the display unit 35 or sound output from the measurement device 30. The control unit 31 may transmit a notification to a nurse station, or the like, via a network such as a hospital information system (HIS) or an electronic medical record (EMR).

For example, the control unit 31 may calculate an index representing a state of a urinary organ such as a kidney on the basis of the oxygen partial pressure and the temperature in the urine and display the index on the display unit 35. The index representing the state of the urinary organ may be calculated by combining information acquired from another device such as a biological information monitor with the oxygen partial pressure and the temperature in the urine. The index is not limited to the index representing the state of the urinary organ and may be an index representing a general condition of the patient. In this case, the control unit 31 implements a function of an index calculation unit that calculates an index representing the state of the urinary organ.

The optical analyzer 52 may output, to the bus, data indicating the characteristics of the fluorescence such as intensity, a phase angle, and decay time of the received fluorescence. In such a case, the control unit 31 calculates a urinary oxygen partial pressure, a urinary oxygen concentration, or the like. The temperature measuring instrument 39 may output data indicating a voltage value of the thermoelectromotive force to the bus. In such a case, the control unit 31 calculates the temperature on the basis of the voltage value.

An optical analysis block including the light source 51, the optical analyzer 52, the light guide path 55, the beam splitter 56, and the first connector 371 may be separate from the measurement device 30. A temperature measurement block including the temperature measuring instrument 39 and the second connector 372 may be separate from the measurement device 30.

In a case where both the optical analysis block and the temperature measurement block are separate bodies, the measurement device 30 of the present embodiment may be configured by combining a general-purpose information processing device such as a personal computer, a tablet, or a smartphone, the optical analysis block, and the temperature measurement block. In such a case, the optical analysis block and the temperature measurement block are connected to the information processing device in a wired or wireless manner.

The display of the display unit 35 illustrated in FIG. 1 is an example. For example, in a case where the light emitter 24 has a phosphor that reacts with potassium ions, the measurement device 30 displays a potassium ion concentration in the urine on the display unit 35 in real time.

The measurement adapter 20 is desirably a single use product supplied to the user in a sterilized state, which can help reduce a risk of occurrence of urinary tract infection.

The measurement adapter 20 may be attached to any tube to be used for continuously discharging a body fluid, or the like, from a patient, such as a chest drainage tube, an abdominal drainage tube, or a brain drainage tube, instead of the bladder indwelling catheter 15. The measurement adapter 20 may be attached to any tube to be used for feeding a liquid into the body of a patient, such as an infusion tube or a feeding tube.

Modification 1-1

The present modification relates to the measurement system 10 that displays time-series data on the display unit 35. Description of parts common to the first embodiment will be omitted.

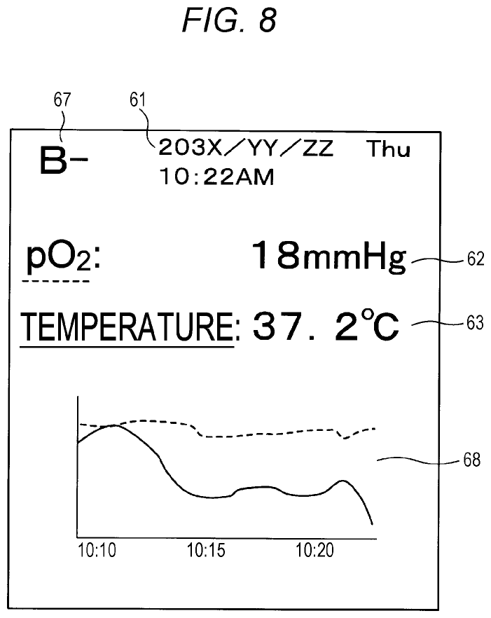

FIG. 8 is a screen example of Modification 1-1. In the present modification, a relatively large display unit 35 can be used. An index field 67, a date and time field 61, an oxygen partial pressure field 62, a temperature field 63, and a graph field 68 are displayed on the screen. In the index field 67, an index representing the state of the kidney is displayed. Combination of the alphabet and the symbol “+” or “−” allows the user to rather easily grasp the state of the kidney of the patient.

The date, the day of the week, and the time are displayed in the date and time field 61. An oxygen partial pressure in urine is displayed in the oxygen partial pressure field 62. A temperature is displayed in the temperature field 63. In the graph field 68, time-series data of an oxygen partial pressure and a temperature in urine are each displayed by a line graph. In FIG. 8, a broken line indicates time-series data of the oxygen partial pressure in urine, and a solid line indicates time-series data of the temperature. A broken line displayed under characters of “pO2” (oxygen partial pressure) and a solid line displayed under characters of “temperature” in the oxygen partial pressure field 62 function as so-called legend fields, which allows the user to rather easily grasp what the graph means.

The line graph illustrated in the graph field 68 is an example of a graph format. Any form of graph that is relatively easy for a user to use in a clinical setting can be used in the graph field 68. For example, in a case where importance is placed on a value per unit time, a bar graph can be used for display in the graph field 68. The user may be able to appropriately specify the format of the graph.

The time-series data may be indicated in a tabular form instead of a graph form. The control unit 31 may appropriately receive setting change of items and a layout to be displayed on the display unit 35 by the user. The user can use the measurement system 10 with relatively user-friendly settings depending on the situation.

Modification 1-2

The present modification relates to the measurement system 10 in which the optical fiber 41 is divided into a fiber for irradiation light and a fiber for light reception. Description of parts common to the first embodiment will be omitted.

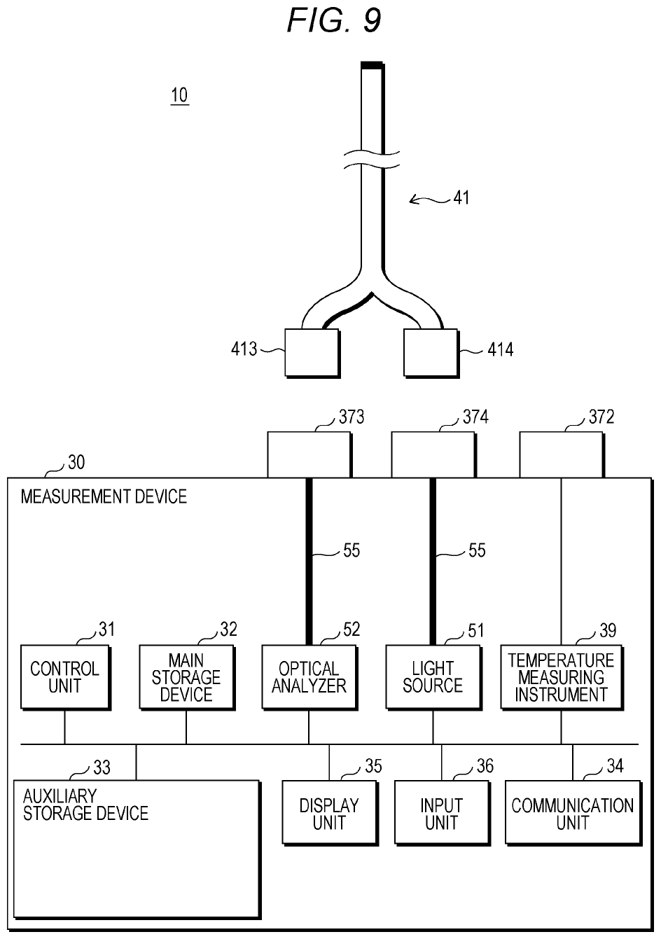

FIG. 9 is an explanatory view illustrating a configuration of Modification 1-2. The optical fiber 41 is divided into two bundles at the end portion, and a fluorescent connector 413 is connected to one bundle and an irradiation light connector 414 is connected to the other bundle.

The measurement device 30 can include a third connector 373 and a fourth connector 374 instead of the first connector 371. The third connector 373 is connected to the optical analyzer 52 via the light guide path 55. The fourth connector 374 is connected to the light source 51 via the light guide path 55.

The light emitter 24 is irradiated with excitation light emitted from the light source 51 through the light guide path 55, the fourth connector 374, and the irradiation light connector 414. The fluorescence emitted from the light emitter 24 enters the optical analyzer 52 via the optical fiber 41, the fluorescent connector 413, the first connector 371, and the light guide path 55.

The fiber for irradiation light and the fiber for light reception may be coupled to one common fiber commonly used for irradiation light (for the excitation light) and light reception (for the fluorescence). The fiber for irradiation light and the fiber for light reception are separate and may be bundled on the side attached to the measurement adapter 20. A fiber having specifications suitable for propagation of excitation light may be used as the fiber for irradiation light, and a fiber having specifications suitable for propagation of fluorescence may be used as the fiber for light reception.

Modification 1-3

The present modification relates to the fastener 49 through which the optical fiber 41 and the thermocouple 45 are inserted. Description of parts common to the first embodiment will be omitted.

FIG. 10 is an explanatory view illustrating a configuration of Modification 1-3. The fastener 49 can include a substantially C-shaped urine collection tube holding portion 496, a round hole-shaped optical fiber holding portion 497, and a thermocouple holding portion 498. The thermocouple 45 is inserted into the thermocouple holding portion 498. The optical fiber 41 and the thermocouple 45 can be inserted through the plurality of fasteners 49.

According to the present modification, it is possible to provide the measurement system 10 capable of smoothly connecting the optical fiber 41 and the thermocouple 45 to the measurement adapter 20 and the measurement device 30 and fixing the optical fiber 41 and the thermocouple 45 by the fastener 49.

Modification 1-4

The present modification relates to the measurement adapter 20 including a cover 29. Description of parts common to the first embodiment will be omitted.

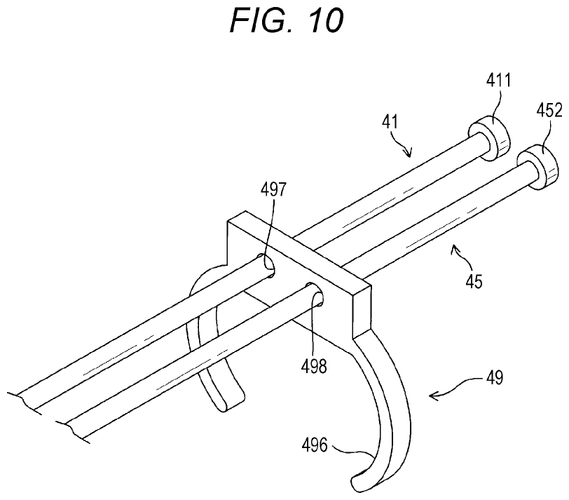

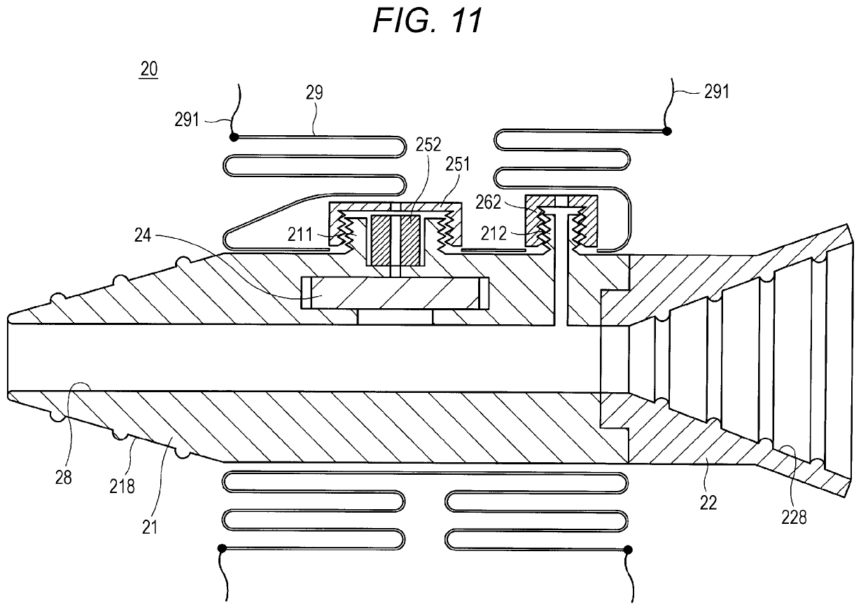

FIG. 11 is an explanatory view for explaining a structure of the measurement adapter 20 of Modification 1-4. The cover 29 is attached to a side surface of the measurement adapter 20. The cover 29 has a tubular shape thicker than the measurement adapter 20 and is attached to the side surface of the measurement adapter 20 with a tack as appropriate. An attachment portion 291 is attached to an end portion of the cover 29. In the present modification, the attachment portion 291 is a string capable of drawing the cover 29.

The first cap 251 and the second cap 262 protrude from holes provided in the cover 29. As illustrated in FIG. 11, the measurement adapter 20 is supplied to the user in a state where the cover 29 is folded a plurality of times.

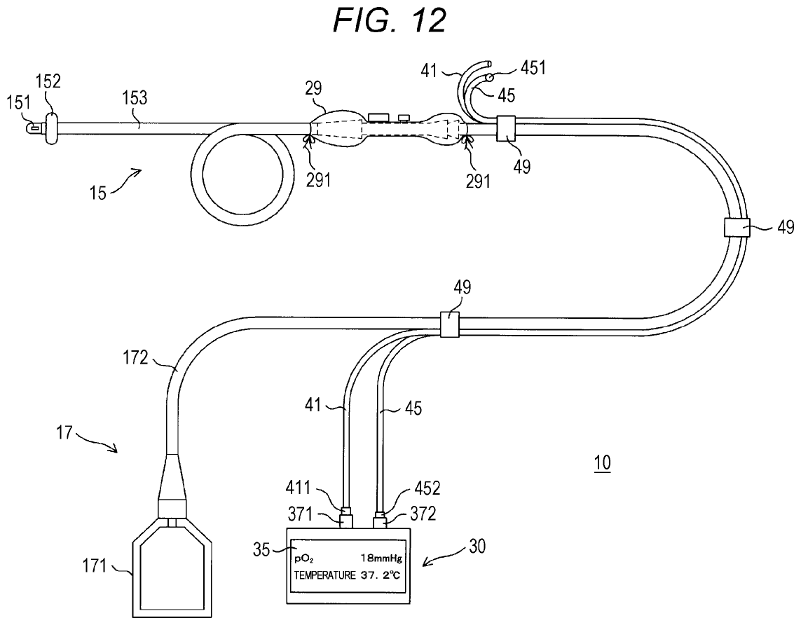

FIG. 12 is an explanatory view illustrating a method of using the measurement system 10 of Modification 1-4. The user connects the bladder indwelling catheter 15, the measurement adapter 20, and the urine collection bag 17. The user connects the optical fiber 41 and the thermocouple 45 to the measurement device 30. The user fixes the optical fiber 41 and the thermocouple 45 to the urine collection tube 172 using the fastener 49.

The user unfolds the folded cover 29 to cover a connection portion between the bladder indwelling catheter 15 and the measurement adapter 20 and a connection portion between the measurement adapter 20 and the urine collection bag 17. The user draws and connects the attachment portion 291. FIG. 12 illustrates a stage where the above steps are completed. The cover 29 may be fixed with a medical tape, or the like, instead of the attachment portion 291.

The attachment portion 291 is not limited to a string capable of drawing the cover 29. For example, the attachment portion 291 may be rubber, or the like, attached to an end portion of the cover 29. The attachment portion 291 may be an adhesive tape attached to the inside of the cover 29. The attachment portion 291 may be made of a material that shrinks into a tubular shape, for example, by heating.

The user then connects the optical fiber 41 and the thermocouple 45 to the measurement adapter 20. As described above, preparation for use of the measurement system 10 is completed.

According to the present modification, it is possible to help reduce a risk of occurrence of urinary tract infection due to invasion of various bacteria, and the like, from the connection portion between the bladder indwelling catheter 15 and the measurement adapter 20 and the connection portion between the measurement adapter 20 and the urine collection bag 17.

The measurement adapter 20 may have a shape in which the first cap 251 and the holding rubber 252 are covered with the cover 29 together with the optical fiber 41 and the thermocouple 45, which can further reduce a risk of occurrence of urinary tract infection.

### Second Embodiment

The present embodiment relates to the measurement system 10 in which the light emitter 24 is attached to an end portion of the optical fiber 41. Description of parts common to the first embodiment will be omitted.

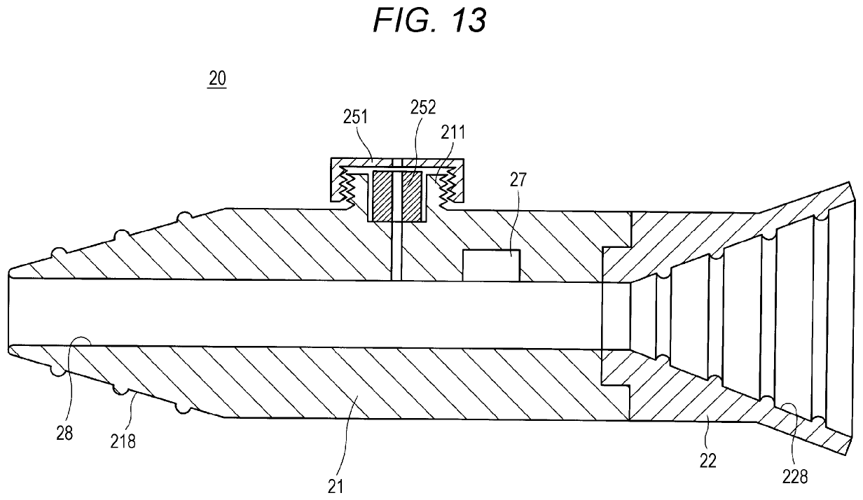

FIG. 13 is an explanatory view illustrating a structure of the measurement adapter 20 according to the second embodiment. The through-hole provided in the first connection portion 211 is not provided with a large-diameter portion for storing the light emitter 24. The measurement adapter 20 does not have the second connection portion 212. The measurement adapter 20 incorporates a flow rate measurement unit 27.

The flow rate measurement unit 27 functions as a flow rate sensor that measures a flow rate of urine flowing through the flow path 28. A portion of the measurement adapter 20 that holds the flow rate measurement unit 27 functions as a flow rate sensor holding portion that holds the flow rate sensor. The flow rate measurement unit 27 can be, for example, an optical flow meter that measures the flow rate by a laser Doppler method using an optical flow rate sensor, or an ultrasonic flow meter that measures the flow rate by an ultrasonic Doppler method using an ultrasonic flow rate sensor. The flow rate measurement unit 27 may be a thermal flow rate sensor. The flow rate measurement unit 27 may measure a urine flow rate by acquiring change over time in weight of the urine that has been conducted. The flow rate measurement unit 27 can have a wireless communication function.

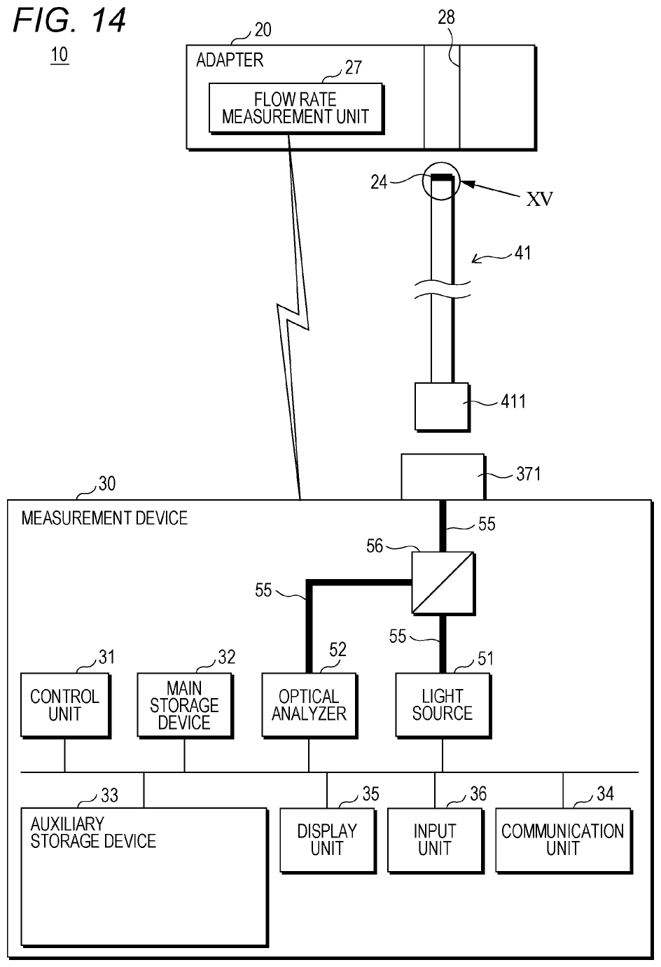

FIG. 14 is an explanatory view illustrating a configuration of the measurement system 10 of the second embodiment. FIG. 14 is a view schematically illustrating each component of the measurement system 10.

The measurement device 30 can include the control unit 31, the main storage device 32, the auxiliary storage device 33, the communication unit 34, the input unit 36, the light source 51, the optical analyzer 52, the light guide path 55, the beam splitter 56, the first connector 371, and the bus. The measurement device 30 receives the flow rate from the flow rate measurement unit 27 via, for example, a wireless communication.

The measurement device 30 may receive the flow rate from the flow rate measurement unit 27 via a wired connection. For example, an ultrasonic Doppler signal may be transmitted from the flow rate measurement unit 27, and the control unit 31 may perform signal analysis to calculate the flow rate.

FIG. 15 is an enlarged view of a portion XV in FIG. 14. The light emitter 24 is adhered to an end surface of the optical fiber 41 via an adhesive layer 241. The light emitter 24 can be, for example, a plate made of a translucent resin into which a phosphor is kneaded. The light emitter 24 may be a translucent plate coated with a phosphor. Furthermore, a light shielding layer that transmits a quencher such as oxygen but shields ambient light may be disposed on the side of the light emitter 24 in contact with liquid. Providing the light shielding layer can help prevent deterioration of the phosphor due to ambient light. The light shielding layer can be made of, for example, carbon black.

Modification 2-1

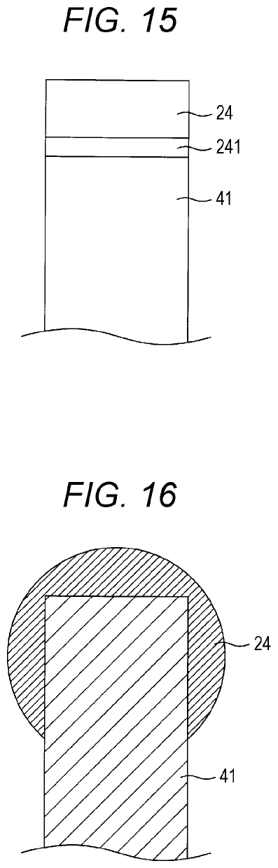

FIG. 16 is a cross-sectional view for explaining a configuration of a distal portion of an optical fiber of Modification 2-1. FIG. 16 illustrates an enlarged view of a portion similar to FIG. 15. In the present modification, the end portion of the optical fiber 41 is covered with the light emitter 24.

For example, the distal end of the optical fiber 41 can be immersed in an uncured transparent resin into which a phosphor is kneaded, pulled up, and then cured, whereby the optical fiber 41 of this modification can be manufactured. A transparent resin into which a phosphor is kneaded may be molded at the distal end of the optical fiber 41 using a mold.

Modification 2-2

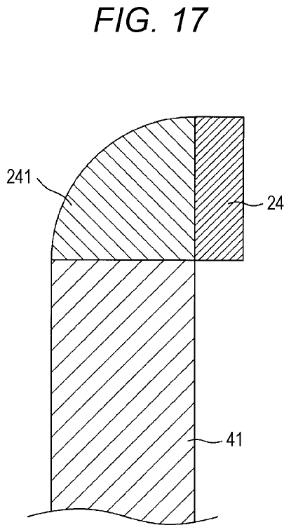

FIG. 17 is a cross-sectional view for explaining a configuration of a distal portion of an optical fiber of Modification 2-2. FIG. 17 illustrates an enlarged view of a portion similar to FIG. 15. In the present modification, the plate-shaped light emitter 24 is fixed substantially perpendicularly to the end portion of the optical fiber 41.

The end portion of the optical fiber 41 and the light emitter 24 are fixed by the adhesive layer 241 using, for example, a translucent resin. Instead of the adhesive layer 241, a translucent component having a predetermined shape may be bonded and fixed between the optical fiber 41 and the light emitter 24.

By fixing the optical fiber 41 to the first connection portion 211 in a state where the light emitter 24 is directed toward the bladder indwelling catheter 15, fresh urine can rather easily touch the light emitter 24, which can provide the measurement system 10 with relatively high sensitivity.

### Third Embodiment

The present embodiment relates to the measurement device 30 including a filter 57 that separates excitation light and fluorescence. Description of parts common to the first embodiment will be omitted.

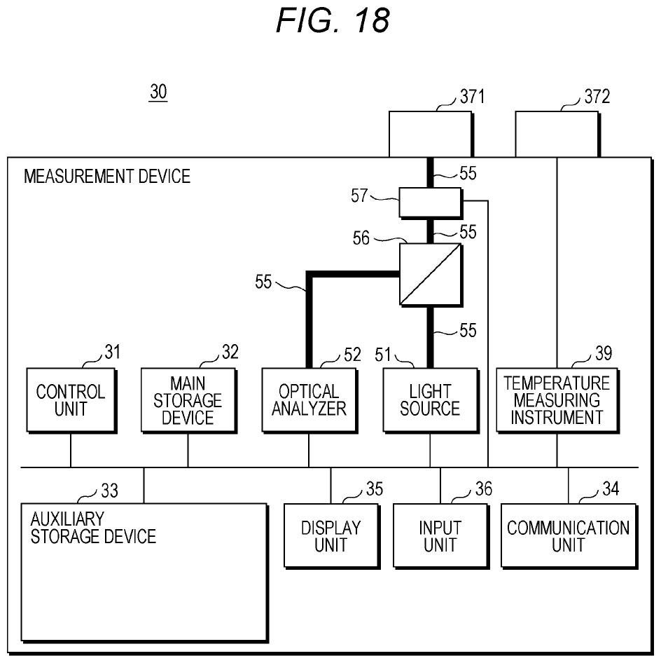

FIG. 18 is an explanatory view for explaining a configuration of the measurement device 30 of the third embodiment. The filter 57 is disposed between the beam splitter 56 and the first connector 371 via the light guide path 55. The control unit 31 can adjust a wavelength range of light transmitted by the filter 57. The light source 51 according to the present embodiment emits broadband light including a wavelength of fluorescence in addition to a wavelength of excitation light. The light source 51 can be, for example, a white LED.

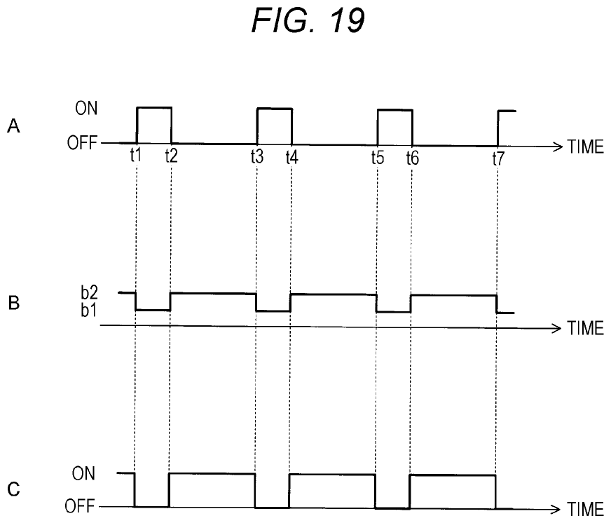

FIGS. 19A to 19C are time charts for explaining operation of the measurement device 30 of the third embodiment. FIG. 19A illustrates timings of turning ON and OFF the light source 51. FIG. 19B illustrates operation timings of the filter 57. b1 indicates that the filter 57 transmits excitation light. b2 indicates that the filter 57 transmits fluorescence. FIG. 19C illustrates timings at which the optical analyzer 52 operates. ON indicates operation of analyzing characteristics of the fluorescence. OFF indicates operation in which the characteristics of the fluorescence are not analyzed. FIGS. 19A to 19C indicate time on horizontal axes.

During a period from time t1 to time t2, the light source 51 is turned ON. During this period, the filter 57 transmits excitation light. The optical analyzer 52 does not operate. The light emitter 24 is irradiated with the excitation light. In a case where the light emitter 24 is in contact with urine, fluorescence corresponding to a state of the urine is emitted.

During a period from time t2 to time t3, the light source 51 is turned OFF. During this period, the filter 57 transmits fluorescence. The optical analyzer 52 analyzes characteristics of the fluorescence and outputs an oxygen partial pressure in the urine to the bus. The same operation is repeated after time t3.

According to the present embodiment, it is possible to provide the measurement system 10 capable of performing accurate measurement even in a case where a wavelength of the fluorescence is included in the light emitted by the light source 51.

### Fourth Embodiment

The present embodiment relates to the measurement system 10 capable of measuring a plurality of items using one light source 51. Description of parts common to the third embodiment will be omitted. The light emitter 24 of the present embodiment includes two types of phosphors. In the following description, two types of phosphors are referred to as a phosphor J and a phosphor K. Wavelengths of fluorescence emitted from the phosphor J and the phosphor K are sufficiently separated from each other.

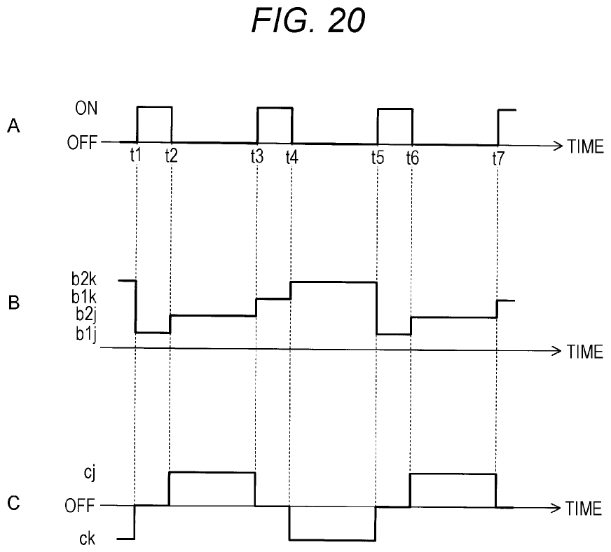

FIGS. 20A to 20C are time charts for explaining operation of the measurement device 30 of the fourth embodiment. FIG. 20A illustrates timings of turning ON and OFF the light source 51. FIG. 20B illustrates operation timings of the filter 57. b1j indicates that the filter 57 transmits excitation light of the phosphor J. b2j indicates that the filter 57 transmits fluorescence emitted by the phosphor J. b1k indicates that the filter 57 transmits excitation light of the phosphor K. b2k indicates that the filter 57 transmits fluorescence emitted by the phosphor K.

FIG. 20C illustrates timings at which the optical analyzer 52 operates. cj represents operation of analyzing characteristics of the fluorescence emitted from the phosphor J. ck indicates operation of analyzing characteristic of the fluorescence emitted by the phosphor K. OFF indicates operation in which the characteristics of the fluorescence are not analyzed. FIGS. 20A to 20C indicate time on horizontal axes.

During a period from time t1 to time t2, the light source 51 is turned ON. During this period, the filter 57 transmits the excitation light of the phosphor J. The optical analyzer 52 does not operate. The light emitter 24 is irradiated with the excitation light. In a case where the light emitter 24 is in contact with urine, the phosphor J emits fluorescence according to a state of the urine.

During a period from time t2 to time t3, the light source 51 is turned OFF. During this period, the filter 57 transmits the fluorescence emitted from the phosphor J. The optical analyzer 52 analyzes characteristics of the fluorescence and outputs items related to the phosphor J to the bus.

During a period from time t3 to time t4, the light source 51 is turned ON. During this period, the filter 57 transmits the excitation light of the phosphor K. The optical analyzer 52 does not operate. The light emitter 24 is irradiated with the excitation light. In a case where the light emitter 24 is in contact with urine, the phosphor K emits fluorescence according to a state of the urine.

During a period from time t4 to time t5, the light source 51 is turned OFF. During this period, the filter 57 transmits the fluorescence emitted from the phosphor K. The optical analyzer 52 analyzes characteristics of the fluorescence and outputs items related to the phosphor K to the bus. The same operation is repeated after time t6.

According to the present embodiment, it is possible to provide the measurement system 10 capable of measuring a plurality of items using one light source 51. The light emitter 24 may have three or more kinds of phosphors. The filter 57 sequentially transmits the excitation light and the fluorescence of each phosphor.

### Fifth Embodiment

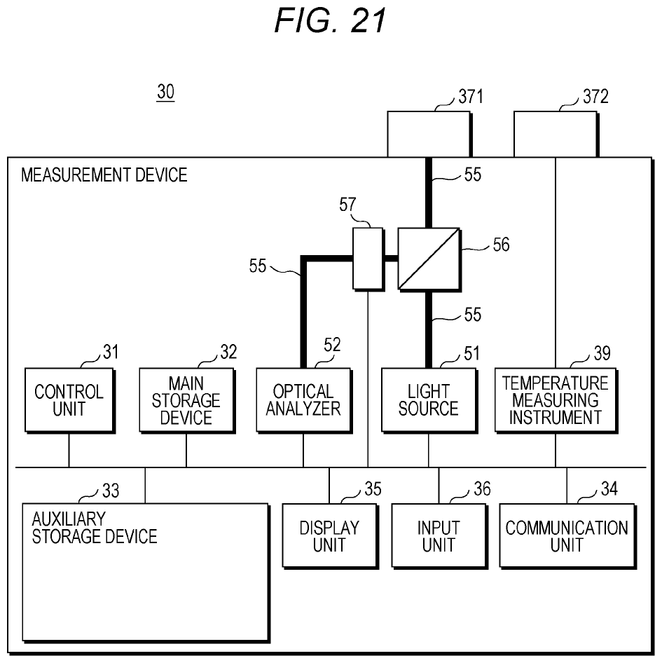

The present embodiment relates to the measurement system 10 using the light source 51 that switches light between a plurality of beams of narrow band excitation light and emits the light. Description of parts common to the third embodiment will be omitted. FIG. 21 is an explanatory view for explaining a configuration of the measurement device 30 of the fifth embodiment. In the present embodiment, the filter 57 is disposed between the beam splitter 56 and the optical analyzer 52.

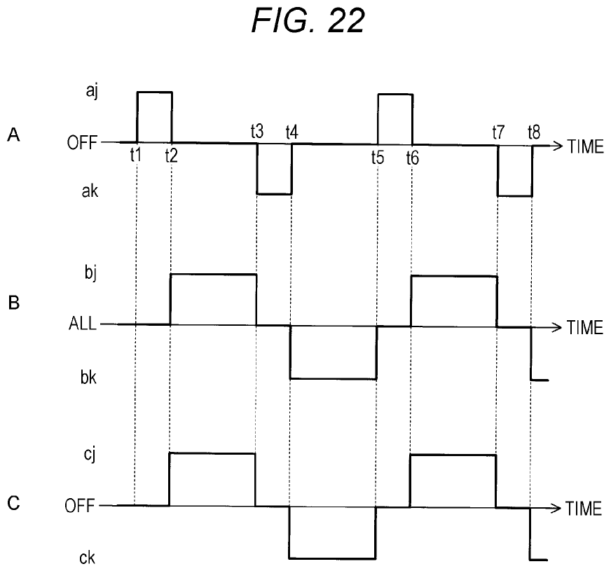

FIGS. 22A to 22C are time charts for explaining operation of the measurement device 30 of the fifth embodiment. FIG. 22A illustrates timings at which the light source 51 operates. aj indicates that the light source 51 emits excitation light of the phosphor J. ak indicates that the light source 51 emits excitation light of the phosphor K.

FIG. 22B illustrates operation timings of the filter 57. ALL indicates that the filter 57 transmits all light. bj indicates that the filter 57 transmits fluorescence emitted by the phosphor J. bk indicates that the filter 57 transmits fluorescence emitted by the phosphor K.

FIG. 22C illustrates timings at which the optical analyzer 52 operates. cj represents operation of analyzing characteristics of the fluorescence emitted from the phosphor J. ck indicates operation of analyzing characteristic of the fluorescence emitted by the phosphor K. OFF indicates operation in which the characteristics of the fluorescence are not analyzed. FIGS. 22A to 22C indicate time on horizontal axes.

During a period from time t1 to time t2, the light source 51 emits excitation light for exciting the phosphor J. During this period, the filter 57 transmits all the light. The optical analyzer 52 does not operate. The light emitter 24 is irradiated with the excitation light. In a case where the light emitter 24 is in contact with urine, the phosphor J emits light according to a state of the urine.

During a period from time t2 to time t3, the light source 51 is turned OFF. During this period, the filter 57 transmits the fluorescence emitted from the phosphor J. The optical analyzer 52 analyzes characteristics of the fluorescence emitted from the phosphor J and outputs the result to the bus.

During a period from time t3 to time t4, the light source 51 emits excitation light for exciting the phosphor K. During this period, the filter 57 transmits all the light. The optical analyzer 52 does not operate. The light emitter 24 is irradiated with the excitation light. In a case where the light emitter 24 is in contact with urine, the phosphor K emits light according to a state of the urine.

During a period from time t4 to time t5, the light source 51 is turned OFF. During this period, the filter 57 transmits the fluorescence emitted from the phosphor K. The optical analyzer 52 analyzes characteristics of the fluorescence emitted from the phosphor K and outputs the result to the bus. The same operation is repeated after time t5.

According to the present embodiment, it is possible to provide the measurement system 10 capable of measuring a plurality of items using one light source 51 and one light emitter 24. The light emitter 24 may have three or more kinds of phosphors. The filter 57 sequentially switches a wavelength of light to be transmitted according to each phosphor.

The light source 51 may emit broadband light including both excitation light of the phosphor J and excitation light of the phosphor K. For example, the light source 51 may emit white light. In such a case, both aj and ak in FIG. 22A indicate that the light source 51 is in an ON state.

### Sixth Embodiment

The present embodiment relates to the measurement device 30 including a plurality of optical analyzers 52. Description of parts common to the fifth embodiment will be omitted.

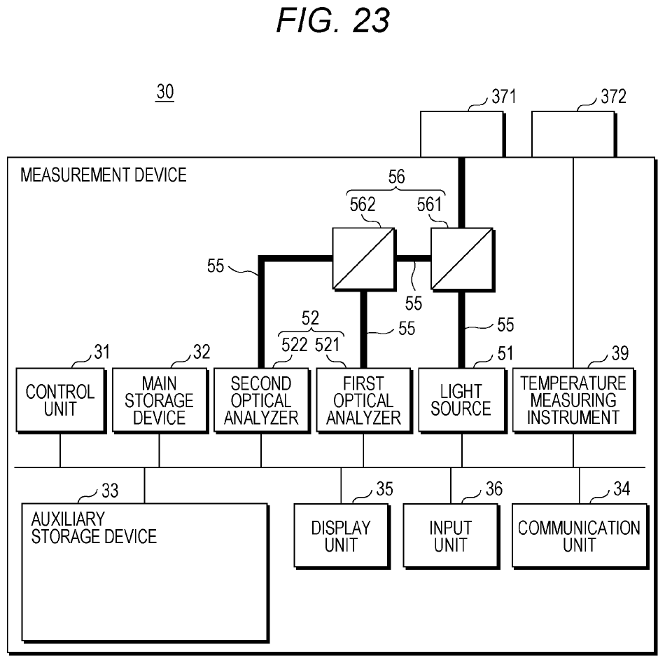

FIG. 23 is an explanatory view illustrating a configuration of the measurement device 30 of the sixth embodiment. The measurement device 30 includes two optical analyzers 52 of a first optical analyzer 521 and a second optical analyzer 522, and two beam splitters 56 of a first beam splitter 561 and a second beam splitter 562.

The first beam splitter 561 is connected between the light source 51 and the first connector 371. The second beam splitter 562 is connected between the first beam splitter 561, and the first optical analyzer 521 and the second optical analyzer 522. The second beam splitter 562 can be, for example, a dichroic beam splitter that separates incident light on the basis of a wavelength. The second beam splitter 562 implements a function of a separation unit that optically separates fluorescence emitted from the plurality of phosphors.

In the following description, a case where the first optical analyzer 521 analyzes the characteristics of the fluorescence emitted from the phosphor J and the second optical analyzer 522 analyzes the characteristics of the fluorescence emitted from the phosphor K will be described as an example. The excitation light capable of exciting both the phosphor J and the phosphor K is emitted from the light source 51.

The excitation light irradiates the light emitter 24 via the light guide path 55, the beam splitter 56, and the optical fiber 41. In a case where the light emitter 24 comes into contact with urine flowing through the flow path 28, the phosphor J and the phosphor K emit fluorescence, respectively.

The fluorescence is incident on the first beam splitter 561 via the optical fiber 41 and the light guide path 55. The fluorescence is incident on the light guide path 55 connected to the second beam splitter 562 by the first beam splitter 561. The fluorescence is separated into the fluorescence emitted from the phosphor J and the other light by the second beam splitter 562. The fluorescence emitted from the phosphor J is incident on the first optical analyzer 521, and the other light is incident on the second optical analyzer 522.

The first optical analyzer 521 analyzes characteristics of the fluorescence emitted from the phosphor J and outputs the result to the bus. The second optical analyzer 522 analyzes characteristics of the fluorescence emitted from the phosphor K and outputs the result to the bus. Note that a filter that transmits only the fluorescence emitted by the phosphor K may be disposed between the second beam splitter 562 and the second optical analyzer 522.

According to the present embodiment, it is possible to provide the measurement system 10 that simultaneously analyzes the fluorescence emitted by two types of phosphors. The light emitter 24 may include three or more types of phosphors, and the measurement device 30 may include the optical analyzer 52 and the beam splitter 56 corresponding to the number of phosphors. Furthermore, in order to adjust to an arbitrary wavelength, an optical filter that passes only a specific wavelength may be disposed in the middle of the light guide path 55.

### Seventh Embodiment

The present embodiment relates to the measurement system 10 in which the measurement device 30 is incorporated in the measurement adapter 20. Description of parts common to the first embodiment will be omitted.

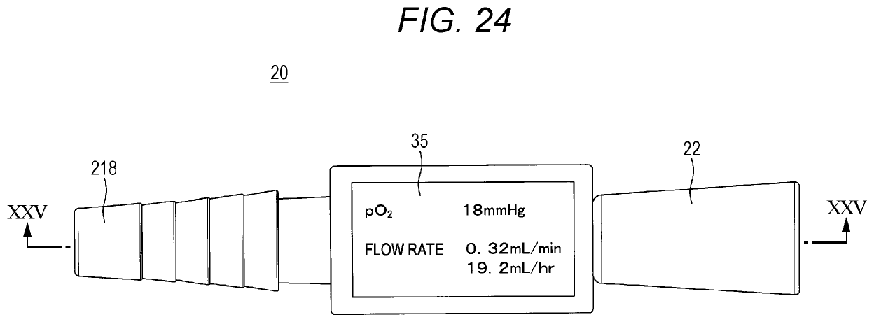

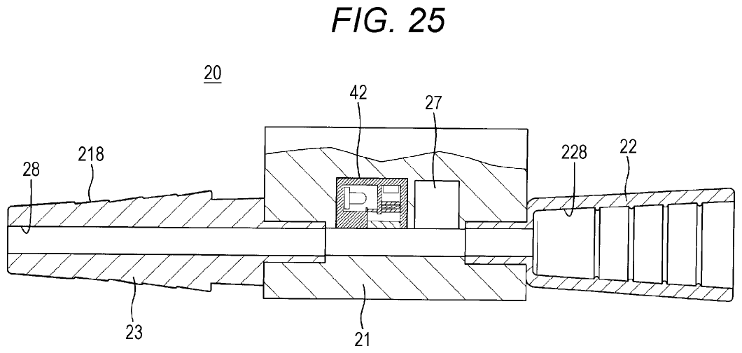

FIG. 24 is a front view of the measurement adapter 20 according to the seventh embodiment. The measurement adapter 20 includes the display unit 35 on a side surface. FIG. 25 is a partial cross-sectional view taken along a line XXV-XXV in FIG. 24.

The measurement adapter 20 includes the first tubular portion 21, the second tubular portion 22, and a third tubular portion 23. The first tubular portion 21, the second tubular portion 22, and the third tubular portion 23 are connected in a longitudinal direction. The flow path 28 is provided along the longitudinal direction of the measurement adapter 20.

The second tubular portion 22 can have a substantially cylindrical shape and has a urine collection bag connection portion 228 on an inner surface of the second tubular portion 22. The third tubular portion 23 has a substantially cylindrical shape and has a catheter connection portion 218 on an outer surface of the third tubular portion 23.

The third tubular portion 23 has a substantially prismatic outer diameter and includes the flow path 28 in the third tubular portion 23. The third tubular portion 23 incorporates the optical sensor unit 42 and the flow rate measurement unit 27. The optical sensor unit 42 and the flow rate measurement unit 27 are disposed to face the flow path 28. In other words, the measurement adapter 20 of the present embodiment includes an optical sensor unit holding portion that holds the optical sensor unit 42 inside the third tubular portion 23 and a flow rate sensor holding portion that holds the flow rate measurement unit 27.

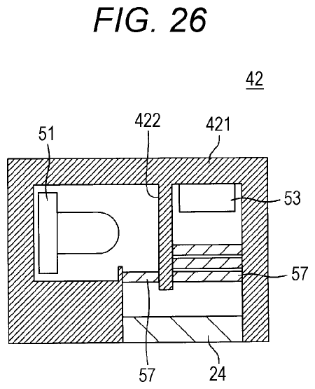

FIG. 26 is an explanatory view illustrating a configuration of the optical sensor unit 42. The optical sensor unit 42 includes a light shielding case 421, the light source 51, a detector 53, the light emitter 24, and a plurality of filters 57. The light shielding case 421 is a chassis of the optical sensor unit 42 and covers a portion other than the light emitter 24. The light shielding case 421 is an example of a light shielding layer covering a portion not facing the flow path of the optical sensor unit 42.

A light shielding partition wall 422 that shields light between the light source 51 and the detector 53 is provided inside the optical sensor unit 42. The light emitter 24 and the optical sensor unit 42 are water-tightly fixed to each other.

The light source 51 can be, for example, a light emitting element such as an LED. The detector 53 is an element that detects light, such as a photodiode. The light source 51 and the detector 53 are controlled by the control unit 31. The optical sensor unit 42 is disposed such that the light emitter 24 touches urine flowing through the flow path 28.

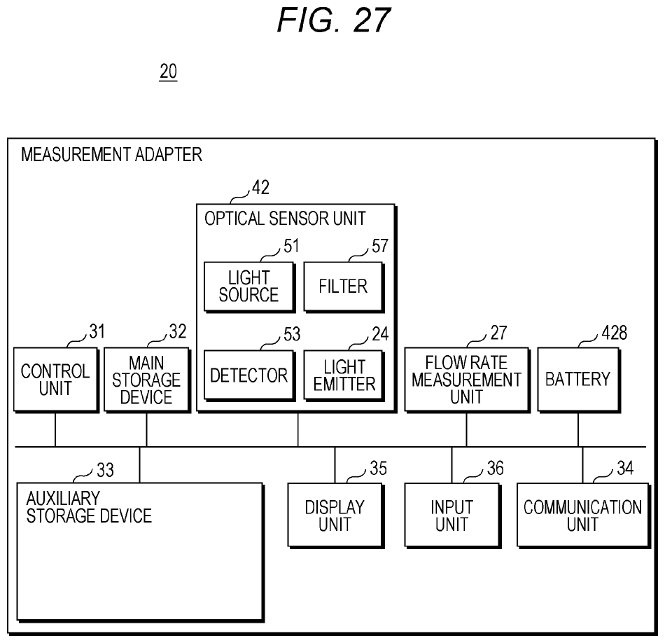

FIG. 27 is an explanatory view for explaining a configuration of the measurement adapter 20 of the seventh embodiment. In addition to the flow rate measurement unit 27, the optical sensor unit 42, and the display unit 35, the measurement adapter 20 includes the control unit 31, the main storage device 32, the auxiliary storage device 33, the communication unit 34, the input unit 36, a battery 428, and a bus.

The control unit 31, the main storage device 32, the auxiliary storage device 33, and the communication unit 34 have functions equivalent to the respective components included in the measurement device 30 described in the first embodiment, and thus, description of the control unit 31, the main storage device 32, the auxiliary storage device 33, and the communication unit 34 will be omitted. As illustrated in FIG. 24, the display unit 35 is attached to a side surface of the first tubular portion 21.

The input unit 36 is a switch attached to the first tubular portion 21. The display unit 35 and the input unit 36 may be stacked to constitute a touch panel. The battery 428 supplies power to each electronic component constituting the measurement adapter 20.

Outline of a method for using the measurement adapter 20 of the present embodiment will be described with reference to FIGS. 24 to 27. The user connects the bladder indwelling catheter 15, the measurement adapter 20, and the urine collection bag 17. The user inserts the shaft 153 into the urethra of the patient. In a state where the distal end of the shaft 153 enters the inside of the bladder, the user inflates the balloon 152. As described above, the bladder indwelling catheter 15 is indwelled in the patient. The urine of the patient passes through the side hole 151, the shaft 153, the flow path 28, and the urine collection tube 172 and is accumulated in the bag 171.

The user operates the input unit 36 to start measurement. Measurement may be automatically started in response to urine flowing through the flow path 28. The light emitted from the light source 51 irradiates the light emitter 24 through the filter 57. In a case where the light emitter 24 comes into contact with urine flowing through the flow path 28, fluorescence is emitted.

The fluorescence is incident on the detector 53 via the filter 57. The detector 53 converts the fluorescence into an electric signal and outputs the electric signal to the bus. The control unit 31 analyzes the electric signal to calculate items related to the phosphor. In other words, the control unit 31 implements the function of the optical analyzer 52 described in the first embodiment in a software manner.

The control unit 31 acquires the flow rate output from the flow rate measurement unit 27 to the bus. The control unit 31 displays items and a flow rate related to the phosphor on the display unit 35. In the example illustrated in FIG. 24, an oxygen partial pressure and a flow rate in urine are displayed on the display unit 35.

According to the present embodiment, it is possible to provide the measurement adapter 20 that can be used stand-alone. The measurement adapter 20 may include a thermometer or other sensors instead of the flow rate measurement unit 27 or together with the flow rate measurement unit 27. The measurement adapter 20 may incorporate a plurality of optical sensor units 42.

The optical sensor unit 42 may include the optical analyzer 52. In such a case, the optical sensor unit 42 outputs the output of the optical analyzer 52 to the bus. The control unit 31 may transmit data to the outside via wireless communication and cause an external display device to display the same items as displayed on the display unit 35.

Modification 7-1

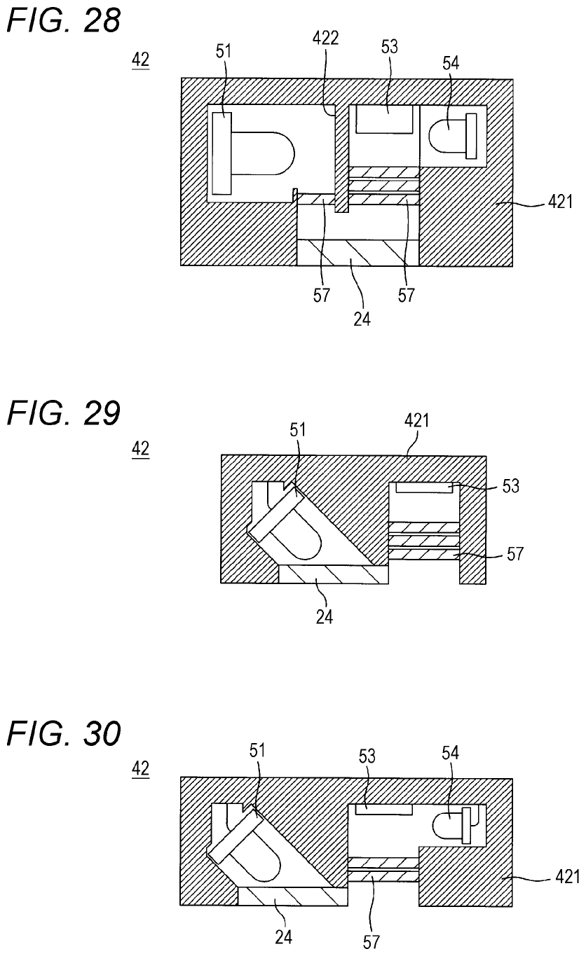

FIG. 28 is an explanatory view illustrating a configuration of the optical sensor unit 42 of Modification 7-1. The optical sensor unit 42 includes a reference light source 54 in the vicinity of the detector 53. The reference light source 54 is disposed at a position where the emitted light is directly incident on the detector 53. By performing calibration or correction using the reference light source 54, it is possible to provide the measurement system 10 with relatively high measurement accuracy.

Modification 7-2

FIG. 29 is an explanatory view illustrating a configuration of the optical sensor unit 42 of Modification 7-2. The light source 51 is disposed such that the optical axis of the excitation light to be emitted forms an angle of about 45 degrees with respect to the longitudinal direction of the flow path 28. The excitation light is emitted from the light source 51 toward the flow path 28 via the light emitter 24. In a case where the light emitter 24 is in contact with urine, the emitted excitation light is incident on the detector 53 via the filter 57.

In the present modification, the light shielding case 421 prevents light in a fluorescence wavelength band included in the light emitted by the light source 51 from reaching the detector 53. It is therefore possible to provide the measurement adapter 20 capable of performing measurement with relatively high accuracy.

Modification 7-3

FIG. 30 is an explanatory view illustrating a configuration of the optical sensor unit 42 of Modification 7-3. The light source 51 is disposed such that the optical axis of the excitation light to be emitted forms an angle of about 45 degrees with respect to the longitudinal direction of the flow path 28. The reference light source 54 is disposed in the vicinity of the detector 53.

Each of the modifications is an example of the arrangement of each component constituting the optical sensor unit 42. The configuration of the optical sensor unit 42 is not limited to the configuration of the sensor unit 42 as disclosed.

Modification 7-4

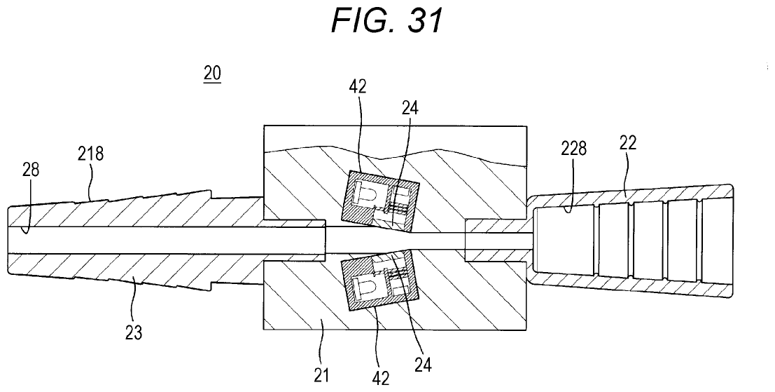

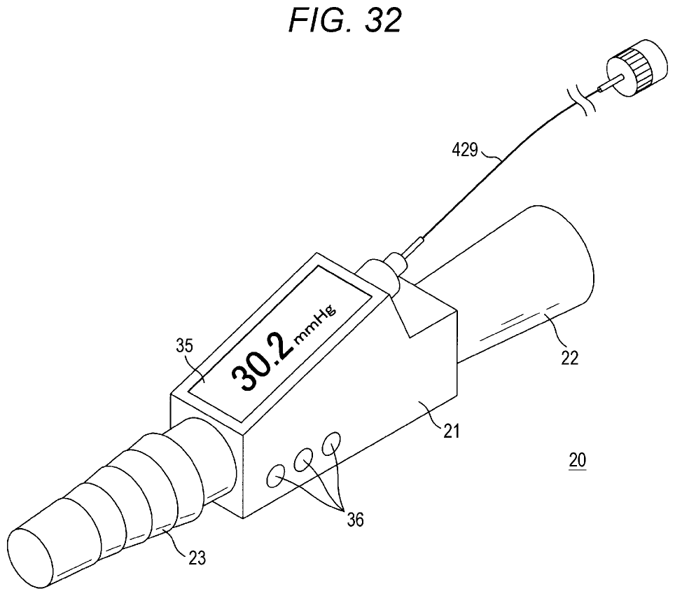

FIG. 31 is a cross-sectional view of the measurement adapter 20 of Modification 7-4. In the present modification, an inner diameter change portion in which an inner diameter is tapered from the catheter connection portion 218 side toward the urine collection bag connection portion 228 side is provided at a central portion of the flow path 28. Two optical sensor units 42 are disposed outside the inner diameter change portion so as to face each other. The light emitter 24 of each optical sensor unit 42 is in contact with the inner diameter change portion.

The inner diameter change portion has, for example, a substantially oval cross section surrounded by two planes perpendicular to a paper surface of FIG. 31 and a cylindrical surface similar to the other portion of the flow path 28. The inner diameter change portion may have a circular cross section. The flow path 28 may have an original thickness on the downstream side of the inner diameter change portion.

By disposing the light emitter 24 in the inner diameter change portion, fresh urine can easily touch the light emitter 24. It is therefore possible to provide the measurement system 10 capable of performing measurement with relatively high accuracy.

The two optical sensor units 42 are arranged to face each other across the flow path 28, and thus, an entire length of the measurement adapter 20 can be kept short even in a case where two optical sensor units 42 are provided.

The optical sensor unit 42 and a sensor that does not use fluorescence, such as a flowmeter or a thermometer, may be arranged to face each other across the flow path 28.

Instead of the optical sensor unit 42, the light emitter 24 described in the first embodiment may be disposed in the inner diameter change portion, and the optical fiber 41 may be attached to the measurement adapter 20. In such a case, it is desirable that the through-hole through which the optical fiber 41 is to be inserted and fixed is substantially perpendicular to the inner diameter change portion, that is, inclined with respect to the flow path 28.

The optical fiber 41 is attached to the measurement adapter 20 in a state of being inclined toward the urine collection bag 17, so that it is possible to provide the measurement adapter 20 in which the optical fiber 41 is relatively easily fixed in a state of being along the bag 171.

Modification 7-5

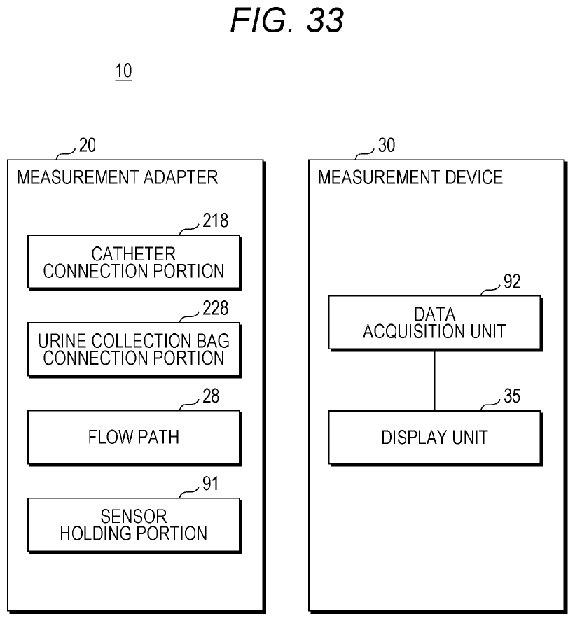

FIG. 32 is a perspective view of the measurement adapter 20 of Modification 7-5. The measurement adapter 20 of the present modification operates by receiving power supply from a power cable 429.

The display unit 35 is disposed obliquely with respect to the long axis of the flow path 28. A connector to which the power cable 429 is to be connected is disposed at an end portion of the display unit 35 on the second tubular portion 22 side. Three input units 36 are disposed on a side surface of the first tubular portion 21. The input unit 36 can be, for example, a power switch, a switch for switching items to be displayed on the display unit 35, a measurement start switch, or the like.

According to the present modification, for example, even in a case where the bladder indwelling catheter 15 is indwelled for a relatively long period of time, for example, such as four weeks, it is possible to provide the measurement adapter 20 for which there is no risk of running out of the battery. Note that power may be supplied to the measurement adapter 20, for example, by a wireless power supply.

The power cable 429 may be a so-called composite cable including the optical fiber 41. In this case, a light guide path for propagating light supplied from the optical fiber 41 to the optical sensor unit 42 is disposed in the measurement adapter 20. It is not necessary to mount a light source such as an LED, so that the optical sensor unit 42 can be made relatively smaller.

### Eighth Embodiment

FIG. 33 is a functional block diagram of the measurement system 10 of the eighth embodiment. The measurement system 10 can include the measurement adapter 20 and the measurement device 30. The measurement adapter 20 includes the catheter connection portion 218 connectable to the bladder indwelling catheter 15, the urine collection bag connection portion 228 connectable to the urine collection bag 17, the flow path 28 disposed between the catheter connection portion 218 and the urine collection bag connection portion 228, and a plurality of sensor holding portions 91 holding sensors 24 and 45 capable of detecting a state of urine flowing in the flow path 28, and the measurement device 30 includes a data acquisition unit 92 that acquires data from the sensors 24 and 45 held by the sensor holding portions 91, and the display unit 35 that displays information on a patient in which the bladder indwelling catheter 15 is indwelled on the basis of the acquired data.

Technical features (components) described in each embodiment can be combined with each other, and new technical features can be formed by the combination.

The detailed description above describes embodiments of a measurement adapter, a measurement system, and a measurement method. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A measurement adapter comprising:
a catheter connection portion configured to be connected to a bladder indwelling catheter;
a urine collection bag connection portion configured to be connected to a urine collection bag;
a flow path disposed between the catheter connection portion and the urine collection bag connection portion; and
a plurality of sensor holding portions configured to hold sensors configured to detect a state of urine flowing in the flow path.

2. The measurement adapter according to claim 1, wherein the sensor holding portions include a light emitter holding portion configured to hold a light emitter, the light emitter configured to emit light upon coming into contact with a component to be measured in the urine flowing through the flow path.

3. The measurement adapter according to claim 2, further comprising:
the light emitter configured to be held by the light emitter holding portion.

4. The measurement adapter according to claim 2, wherein
the sensor holding portions include an optical fiber holding portion configured to hold an optical fiber, the optical fiber configured to propagate irradiation light with which the light emitter is to be irradiated and radiation light emitted from the light emitter, the radiation light being fluorescence; and
the optical fiber held by the optical fiber holding portion.

5. The measurement adapter according to claim 1,
wherein the sensor holding portions include an optical sensor unit holding portion configured to hold an optical sensor unit, and
the optical sensor unit includes:
a light emitter configured to emit light upon coming into contact with a component to be measured in the urine flowing through the flow path;
a light source configured to irradiate the light emitter with light;
a detector configured to detect radiation light emitted from the light emitter; and a light shielding layer configured to cover a portion not facing the flow path.

6. The measurement adapter according to claim 2, wherein
the light emitter includes a plurality of phosphors configured to react with each of a plurality of components in urine to emit fluorescence; and
the fluorescence emitted from the phosphor configured to change according to an oxygen partial pressure in urine, a carbon dioxide partial pressure in urine, a hydrogen ion index in urine, an ion concentration in urine, potassium ions in urine, sodium ions in urine, or a flow rate of urine.

7. The measurement adapter according to claim 1, wherein
the sensor holding portions include a temperature sensor holding portion configured to hold a temperature sensor, the temperature sensor configured to measure a temperature of the urine flowing through the flow path; and
a flow rate sensor holding portion configured to hold a flow rate sensor, the flow rate sensor configured to measure a flow rate of the urine flowing through the flow path, and wherein the flow rate sensor is any of an optical flow rate sensor, an ultrasonic flow rate sensor, a thermal flow rate sensor, or a sensor configured to measure a urine flow rate on the basis of change in weight of urine that has been conducted.

8. The measurement adapter according to claim 1, further comprising:
a plurality of the sensor holding portions arranged along a longitudinal direction of the flow path;
two of a plurality of the sensor holding portions are arranged to face each other across the flow path;
a check valve configured to prevent backflow in the flow path is provided on a side closer to the catheter connection portion than any of a plurality of the sensor holding portions.
a first material forming the catheter connection portion is a material different from a second material forming the urine collection bag connection portion; and
the first material is softer than the second material, and the second material is elastomer or rubber.

9. The measurement adapter according to claim 1, wherein
the flow path includes an inner diameter change portion whose inner diameter decreases from the catheter connection portion side toward the urine collection bag connection portion side; and
the sensor holding portions are disposed in the inner diameter change portion.

10. A measurement system comprising:
a measurement adapter, the measurement adapter including:
a catheter connection portion configured to be connectable to a bladder indwelling catheter;
a urine collection bag connection portion configured to be connectable to a urine collection bag;
a flow path disposed between the catheter connection portion and the urine collection bag connection portion; and
a plurality of sensor holding portions configured to hold sensors, the sensors configured to detect a state of urine flowing in the flow path; and
a measurement device, the measurement device including:
a data acquisition unit configured to acquire data from the sensors held by the sensor holding portions; and
a display unit configured to display information on a patient in which the bladder indwelling catheter is indwelled on the basis of the acquired data.

11. The measurement system according to claim 10, wherein
the sensor holding portions are configured to hold a light emitter, the light emitter configured to emit light upon coming into contact with a component to be measured in the urine flowing through the flow path; and
the data acquisition unit is configured to acquire data related to a light emission state of the light emitter.

12. The measurement system according to claim 11,
wherein the light emitter includes a phosphor configured to react with a component in urine to emit fluorescence;
the measurement device includes a light source configured to irradiate the light emitter with excitation light; and
the data acquisition unit is configured to acquired data related to the fluorescence emitted by the phosphor.

13. The measurement system according to claim 12, wherein
the light emitter includes a plurality of the phosphors configured to react with each of a plurality of components in urine to emit fluorescence;
the measurement device includes a separation unit configured to optically separate the fluorescence emitted from each of the phosphors; and
the data acquisition unit is configured to acquire data related to each fluorescence separated by the separation unit.

14. The measurement system according to claim 13, wherein the fluorescence emitted from the phosphor is configured to change according to an oxygen partial pressure in urine, a carbon dioxide partial pressure in urine, a hydrogen ion index in urine, an ion concentration in urine, potassium ions in urine, sodium ions in urine, or a flow rate of urine.

15. The measurement system according to claim 12, further comprising:
an optical fiber configured to propagate excitation light from the light source to the light emitter; and
wherein the sensor holding portions include an optical fiber holding portion configured to hold the optical fiber.

16. The measurement system according to claim 15, further comprising:
a fastener configured to fasten the optical fiber to a surface of the urine collection bag, the fastener being configured to fasten a cable connected to the sensors disposed in the sensor holding portions to the surface of the urine collection bag;
wherein the light emitter is fixed to the sensor holding portions;
the optical fiber is detachable from the optical fiber holding portion; and
wherein the light emitter is fixed to an end portion of the optical fiber.

17. The measurement system according to claim 11,
wherein the sensor holding portions include an optical sensor unit holding portion configured to hold an optical sensor unit; and
the optical sensor unit includes:
the light emitter;
a light source configured to irradiate the light emitter with light; and
a detector configured to detect radiation light emitted from the light emitter.

18. The measurement system according to claim 17, wherein
the optical sensor unit includes a light shielding layer configured to cover a portion not facing the flow path;
the light emitter includes a phosphor configured to react with a component in urine to emit fluorescence;
the light source is configured to irradiate the light emitter with excitation light,
the detector is configured to detect the fluorescence emitted from the phosphor;
the data acquisition unit is configured to acquire data from the detector via wireless communication; and
wherein the measurement device is integrally with the measurement adapter.

19. The measurement system according to claim 10, further comprising:
a temperature sensor configured to be held by the sensor holding portions, the temperature sensor configured to measure a temperature of the urine flowing through the flow path;
a flow rate sensor configured to be held by the sensor holding portions, the flow rate sensor configured to measure a flow rate of the urine flowing through the flow path;
the display unit configured to display information on a patient in time series;
the measurement device including an index calculation unit configured to calculate an index representing a state of kidney on the basis of data acquired by the data acquisition unit from each of a plurality of sensors, and
the display unit is configured to display the index calculated by the index calculation unit.

20. A measurement method comprising:
acquiring data acquired using sensors from a measurement adapter connected between a bladder indwelling catheter and a urine collection bag, the measurement adapter including a flow path through which urine flows from the bladder indwelling catheter to the urine collection bag, and a plurality of sensor holding portions that hold the sensors that detect a state of the urine flowing in the flow path; and
displaying information on a state of a urinary organ of a patient in which the bladder indwelling catheter is indwelled on the basis of the acquired data.
